(19) United States
(12) Patent Application Publication
(10) Pub. No.: US 2024/0299164 A1
(43) Pub. Date: Sep. 12, 2024

(54) PROSTHETIC VALVE DELIVERY APPARATUS WITH STRAIN RELIEF NOSECONE

(71) Applicant: EDWARDS LIFESCIENCES CORPORATION, Irvine, CA (US)

(72) Inventors: Jose Luis Lopez, Fountain Valley, CA (US); Salomon Xavier Valencia, Aliso Viejo, CA (US); Brad Buzea, Trabuco Canyon, CA (US)

(21) Appl. No.: 18/663,486

(22) Filed: May 14, 2024

Related U.S. Application Data
(63) Continuation of application No. PCT/US2022/049979, filed on Nov. 15, 2022.
(60) Provisional application No. 63/280,245, filed on Nov. 17, 2021.

(51) Int. Cl. A61F 2/24

(52) U.S. Cl. CPC A61F 2/2418; A61F 2/243

(57) ABSTRACT

A delivery apparatus for an expandable prosthetic heart valve can include a handle, a shaft having a proximal portion and a distal portion, the proximal portion being coupled to the handle, and a nosecone coupled to the distal portion of the shaft. The nosecone can include a proximal portion and a distal portion coupled to the proximal portion. The distal portion can be configured to flexibly curve relative to the proximal portion and the proximal portion is formed of a first material that is relatively harder than a second material forming the distal portion.

### CROSS REFERENCE TO RELATED APPLICATIONS

The present application is a continuation of PCT/US2022/049979, filed Nov. 15, 2022, which claims the benefit of U.S. Provisional Application No. 63/280,245, filed Nov. 17, 2021, which both of applications are incorporated herein by reference in their entireties.

### FIELD

The present disclosure concerns examples of prosthetic valve delivery assemblies and related methods.

### BACKGROUND

Endovascular delivery devices are used in various procedures to deliver prosthetic medical devices or instruments to locations inside the body that are not readily accessible by surgery or where access without surgery is desirable. Access to a target location inside the body can be achieved by inserting and guiding the delivery device through a pathway or lumen in the body, including, but not limited to, a blood vessel, an esophagus, a trachea, any portion of the gastrointestinal tract, a lymphatic vessel, to name a few. In one specific example, a prosthetic heart valve can be mounted in a crimped state on the distal end of a delivery device and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, by actuating mechanical actuators of the prosthetic valve, or by deploying the prosthetic valve from a sheath of the delivery device so that the prosthetic valve can self-expand to its functional size. Despite the recent advancements in percutaneous valve technology, there remains a need for improved systems and methods for delivery of such valves.

### SUMMARY

Described herein are prosthetic valve delivery assemblies and related methods, which can be used to deliver a prosthetic valve to a location within a body of a subject. In some implementations, the prosthetic valve delivery assemblies can be used to deliver a medical device through the vasculature, such as to a heart of the subject.

A delivery apparatus for an expandable prosthetic heart valve or another type of implantable medical device can comprise a handle and one or more shafts coupled to the handle.

In some examples, a delivery apparatus can comprise a nosecone coupled to a distal portion of a shaft.

In some examples, a delivery apparatus can comprise a nosecone comprising a proximal portion and a distal portion coupled to the proximal portion and configured to flexibly curve relative to the proximal portion.

In some examples, a delivery apparatus can comprise a nosecone comprising a distal portion and a proximal portion coupled to the distal portion and formed of a first material that is relatively harder than a second material forming the distal portion.

In some examples, a delivery apparatus can comprise a delivery capsule configured to retain an expandable prosthetic heart valve in a radially-compressed configuration for delivery into a patient.

In some examples, a delivery apparatus can comprise a delivery capsule comprising a main body and a tapered-distal tip portion axially extending from the main body and tapering in a distal direction.

In some examples, a delivery apparatus can comprise a delivery capsule comprising a main body and a tapered-distal tip portion, the tapered-distal tip portion formed from a material having a hardness less than a material forming the main body.

In some examples, a delivery apparatus can comprise a delivery capsule comprising a main body and a tapered-distal tip portion extending circumferentially around a distal portion of the main body.

In some examples, a delivery apparatus comprises a handle, a shaft having a proximal portion and a distal portion, the proximal portion being coupled to the handle, and a nosecone coupled to the distal portion of the shaft, the nosecone comprising a proximal portion and a distal portion coupled to the proximal portion, wherein the distal portion is configured to flexibly curve relative to the proximal portion and the proximal portion is formed of a first material that is relatively harder than a second material forming the distal portion.

In some examples, a delivery apparatus for an implantable medical device comprises a handle, a shaft having a proximal portion and a distal portion, the proximal portion being coupled to the handle, and a nosecone coupled to the distal portion of the shaft. The nosecone comprises a proximal portion and a distal portion coupled to the proximal portion, wherein the distal portion is configured to bend relative to the proximal portion. The proximal portion is formed of a first material that is relatively harder than a second material forming the distal portion.

In some examples, a delivery apparatus for an expandable prosthetic heart valve comprises a handle, and a first shaft and a second shaft extending over the first shaft, each shaft comprising a distal portion and a proximal portion coupled to the handle. The distal portion of the second shaft is coupled to a delivery capsule of the delivery apparatus. The delivery capsule comprises a main body configured to retain an expandable prosthetic heart valve in a radially-compressed configuration for delivery into a patient and a tapered-distal tip portion axially extending from the main body and tapering in a distal direction.

In some examples, a delivery apparatus for an expandable prosthetic heart valve comprises a handle, a first shaft and a second shaft extending over the first shaft, each shaft comprising a distal portion and a proximal portion coupled to the handle, the distal portion of the second shaft coupled to a delivery capsule of the delivery apparatus. The delivery capsule comprises a main body configured to retain an expandable prosthetic heart valve in a radially-compressed configuration for delivery into a patient and a distal rim axially extending from the main body. The delivery apparatus further comprises a nosecone coupled to the distal portion of the first shaft, the nosecone comprising a proximal portion and a distal portion coupled to the proximal portion and configured to bend relative to the proximal portion and the delivery capsule, wherein the delivery capsule is movable between a closed position and an open position. When delivery capsule is in the closed position, the main body of the delivery capsule receives the proximal portion of the nosecone, and the distal rim of delivery capsule extends over the proximal portion of the nosecone and partially over the distal portion of the nosecone. When the delivery capsule is in the open position, the distal rim is spaced proximally from the nosecone.

In some examples, a delivery apparatus for an expandable prosthetic heart valve comprises a handle, a first shaft having a distal portion and proximal portion coupled to the handle, a nosecone coupled to the distal portion of the first shaft, the nosecone comprising a proximal portion and a distal portion coupled to the proximal portion by a joint comprising a projection received in a cavity. The distal portion of the nosecone comprises a plurality of circumferential grooves along a portion of the distal portion and the proximal portion is formed of a first material that is relatively harder than a second material forming the distal portion. The delivery apparatus further comprises a second shaft extending over the first shaft and a delivery capsule coupled to a distal end portion of the second shaft, wherein the delivery capsule comprises a tapered-distal tip portion and a main body configured to receive the proximal portion of the nosecone and retain an expandable prosthetic heart valve in a radially-compressed configuration when the delivery capsule is in a closed position. The tapered-distal tip portion is flexible relative to the main body and configured to extend over one or more of the circumferential grooves of the distal portion and an midsection of the nosecone formed at an interface of the proximal portion and the distal portion of the nosecone when the delivery capsule is in the closed position.

In some examples, a method for delivering an expandable prosthetic heart valve comprises advancing a delivery apparatus into a native vasculature of a patient, wherein the delivery apparatus comprises a nosecone coupled to a distal end portion of a shaft and an expandable prosthetic heart valve mounted in a radially-compressed configuration around the distal end portion of the shaft. The nosecone comprises proximal portion and a distal portion coupled to the proximal portion. The distal portion comprises a first material and the proximal portion comprises a second material, wherein the first material is softer than the second material. The method further comprises urging a distal portion of the nosecone against a vasculature wall of the patient to cause the distal portion to bend relative to the proximal portion.

In some examples, a method for delivering an expandable prosthetic heart valve comprises advancing into a native vasculature of a patient a delivery apparatus comprising an expandable prosthetic heart valve mounted in a radially-compressed configuration around a distal portion of a first shaft and retained in a delivery capsule of a second shaft extending over the first shaft, such that a distal portion of a nosecone of the delivery apparatus contacts a vasculature wall of the patient. Contact between the nosecone and the vasculature wall of the patient causes the distal portion of the nosecone to flexibly curve relative to a proximal portion of the nosecone. The delivery capsule comprises a tapered-distal tip extending over a portion of the distal portion of the nosecone such that the tapered-distal tip minimizes formation of a gap between the nosecone and delivery capsule as the distal portion flexibly curves.

In some examples, a method for implanting an expandable prosthetic heart valve into an aortic annulus of a patient comprises advancing an expandable prosthetic heart valve and a distal portion of a first shaft and a second shaft of a delivery apparatus into an aorta of the patient such that a distal portion of a nosecone of the delivery apparatus contacts a wall of the aorta, wherein the expandable prosthetic heart valve is mounted in a radially-compressed configuration around a distal portion of the first shaft and retained within a delivery capsule along a distal section of the second shaft. A proximal portion of the nosecone is coupled to the distal portion of the first shaft and a distal rim of the delivery capsule extends over a portion of the distal portion of the nosecone. Contact between the nosecone and the wall of the aorta causes the distal portion of the nosecone to flexibly curve relative to the proximal portion of the nosecone and the delivery capsule, and the distal rim minimizes formation of a gap between the nosecone and the delivery capsule as the distal portion of the nosecone flexibly curves. The method further comprises inserting the delivery capsule and the expandable prosthetic heart valve into a native annulus of the patient such that the nosecone extends through the aortic annulus and into a left ventricle of the patient and retracting the second shaft, and expanding the prosthetic heart valve from a radially-compressed configuration to a radially-expanded configuration within the native annulus.

In some examples, an expandable prosthetic heart valve delivery assembly comprises a delivery apparatus comprising a handle, a shaft comprising a distal portion and a proximal portion coupled to the handle, and a nosecone coupled to the distal portion of the shaft. The nosecone comprising a proximal portion and a distal portion coupled to the proximal portion. The delivery assembly further comprises an expandable prosthetic heart valve mounted in a radially-compressed configuration around the distal portion of the shaft. The proximal portion of the nosecone is formed of a first material that is relatively harder than a second material forming the distal portion, and the distal portion of the nosecone is configured to flexibly curve relative the proximal portion of the nosecone and the expandable prosthetic heart valve.

In some examples, an expandable prosthetic heart valve delivery assembly comprises a delivery apparatus comprising a handle, a shaft comprising a distal portion and a proximal portion coupled to the handle, and a nosecone coupled to the distal portion of the shaft, the nosecone comprising a proximal portion and a distal portion coupled to the proximal portion; and an expandable prosthetic heart valve mounted in a radially-compressed configuration around the distal portion of the shaft. The distal portion of the nosecone comprises a material relatively softer than a material of the proximal portion and a plurality of circumferential grooves aligned coaxially along the distal portion of the nosecone such that the body of the distal portion is configured to flexibly curve relative to the proximal portion of the nosecone.

In some examples, an expandable prosthetic heart valve delivery assembly comprises a delivery apparatus comprising a handle, a shaft comprising a distal portion and a proximal portion coupled to the handle, and a nosecone coupled to the distal portion of the shaft, the nosecone comprising a proximal portion and a distal portion coupled to the proximal portion by a projection received in a cavity. The proximal portion is formed of a first material that is relatively harder than a second material forming the distal portion. The delivery assembly further comprises an expandable prosthetic heart valve mounted in a radially-compressed configuration around the distal portion of the shaft. The distal portion of the nosecone is configured to flexibly curve relative the proximal portion of the nosecone, and one of the proximal portion and the distal portion comprises the projection and the other of the proximal portion and the distal portion comprises the cavity.

In some examples, an expandable prosthetic heart valve delivery assembly comprises a delivery apparatus comprising a handle, a first shaft, and a second shaft extending over the first shaft, each shaft comprising a distal portion and a proximal portion coupled to the handle, the distal portion of the second shaft having a delivery capsule comprising a main body and a tapered-distal tip axially extending from the main body. The delivery assembly further comprises an expandable prosthetic heart valve mounted in a radially-compressed configuration around the distal portion of the first shaft and within the delivery capsule of the second shaft. The tapered-distal tip of the delivery capsule extends circumferentially around a tapered portion of a nosecone coupled to the distal portion of the first shaft.

In some examples, an expandable prosthetic heart valve delivery assembly comprises a delivery apparatus comprising a handle, a nosecone, a first shaft, and a second shaft extending over the first shaft, each shaft comprising a distal portion and a proximal portion coupled to the handle. The nosecone being coupled to the distal portion of the first shaft and the second shaft having a delivery capsule comprising a main body and a tapered-distal tip axially extending from the main body. The delivery assembly further comprises an expandable prosthetic heart valve mounted in a radially-compressed configuration around the distal portion of the first shaft and within the delivery capsule of the second shaft. The nosecone comprises a proximal portion and a distal portion coupled to the proximal portion, the proximal portion being received by the delivery capsule and the distal portion of the nosecone being configured to flexibly curve relative the proximal portion of the nosecone and the expandable prosthetic heart valve.

In some examples, an expandable prosthetic heart valve delivery assembly comprises a delivery apparatus comprising a handle, a nosecone a first shaft, and a second shaft extending over the first shaft, each shaft comprising a distal portion and a proximal portion coupled to the handle. The nosecone being coupled to the distal portion of the first shaft, and the second shaft having a delivery capsule comprising a main body and a tapered-distal tip axially extending from the main body. The delivery assembly further comprises an expandable prosthetic heart valve mounted in a radially-compressed configuration around the distal portion of the first shaft and within the delivery capsule of the second shaft. The nosecone comprises a distal portion, a proximal portion coupled to the distal portion, and a flush port extending along and through the surface of the distal portion and the proximal portion, the distal portion of the nosecone being configured to flexibly curve relative to the proximal portion and the delivery capsule and the flush port being configured to allow fluid to flow therethrough. The tapered-distal tip is configured to extend partially over the distal portion, the proximal portion, and the flush port of the nosecone.

In some examples, a delivery apparatus or delivery assembly comprises one or more of the components recited in Examples 1-71 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a delivery assembly comprising a mechanically expandable prosthetic heart valve and a delivery apparatus.

FIG. 2 is a perspective view of the prosthetic heart valve of FIG. 1.

FIG. 3 is a side view of a nosecone of the delivery apparatus of FIG. 1 in a straight state and according to a first example.

FIG. 4 is a cross-sectional side view of the nosecone of FIG. 3

FIG. 5 is a side view of the nosecone from FIGS. 3-4 in a curved state.

FIG. 6 is a cross-sectional side view of the nosecone from FIG. 5.

FIG. 7 is a perspective view of a delivery capsule and a nosecone according to a second example.

FIG. 8 is a perspective view of the delivery capsule receiving and butting against a proximal portion of the nosecone from FIG. 7.

FIG. 9 is a perspective view of a delivery capsule, according to one example, including a tapered-distal tip.

FIG. 10 is a cross-sectional side view of the delivery capsule from FIG. 9.

FIG. 11 is a perspective view of the delivery capsule of FIG. 9 and a nosecone according to a third example.

FIG. 12 is a perspective view of the delivery capsule and nosecone of FIG. 11, where the tapered-distal tip of the delivery capsule extends partially over the nosecone.

FIG. 13 is a cross-sectional, perspective view of the delivery capsule and nosecone of FIG. 12.

FIG. 14 is perspective view of a distal end portion of the delivery assembly comprising the prosthetic heart valve of FIG. 1, the nosecone of FIG. 11, and delivery capsule of FIG. 9, depicting the prosthetic heart valve partially disposed within the delivery capsule.

FIG. 15 is a side view of a distal end portion of the delivery assembly of FIG. 14, depicting the prosthetic heart valve fully disposed within the delivery capsule.

FIGS. 16-22 depict various steps of an implantation procedure in which the delivery assembly of FIG. 15 is used.

### DETAILED DESCRIPTION

### General Considerations

It should be understood that the disclosed examples can be adapted for delivering and implanting prosthetic heart valves in any of the native annuluses of the heart (e.g., the aortic, pulmonary, mitral, and tricuspid annuluses), and can be used with any of the various delivery devices for delivering the prosthetic heart valve using any of a number of delivery approaches (e.g., retrograde, antegrade, transseptal, transseptal, transventricular, transatrial, etc.). Although the examples of delivery apparatuses disclosed herein are described in the context of being to implant a prosthetic heart valve, the delivery apparatuses can be used to deliver and implant any of various medical implants within the body, including, but not limited to, venous valves, stents, grafts, heart valve repair devices, etc.

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not excluded the presence of intermediate elements between the coupled or associated items absent specific contrary language.

As used in this application, the term “and/or” used between the last two of a list of elements any one or more of the listed elements. For example, the phrase “A, B, and/or C” means “A,” “B,” “C,” “A and B,” “A and C,” “B and C,” or “A, B, and C.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while the distal motion of the device is motion of the device away from the user and toward the implantation site (e.g., into the patient's body). The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such has the longitudinal axis of the prosthetic heart valve).

### Examples of the Disclosed Technology

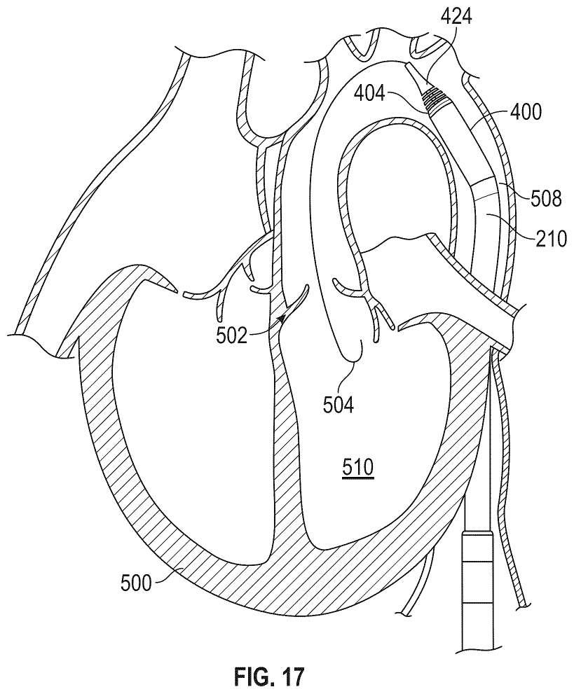

Prosthetic devices (for example, stents and prosthetic valves) and the components of a delivery device can render a delivery assembly inflexible during an implantation procedure. For example, the length and rigidity of a nosecone and delivery capsule, and the prosthetic device therein, may result in the nosecone and delivery capsule being pushed axially against the inner vasculature wall of a patient. In particular, during implantation of the prosthetic device, the delivery apparatus might be pushed axially against the vessel wall as the prosthetic device is maneuvered through the vessel's curvature, such as through relatively sharp curves of a native aortic arch (for example, as shown in FIGS. 17-23). This axial force may result in undesirable disruption to the vessel wall and separation between the delivery capsule and the nosecone of the delivery apparatus. For example, as a delivery device is advanced around a native arch, the nosecone may twist and/or rotate relative to the delivery capsule as the nosecone contacts the surrounding vessel wall. As the nosecone twists and rotates against the delivery capsule, it can apply a force against the inner wall of the delivery capsule and cause separation between the two components. This separation can potentially lead to unwanted leakage into the delivery capsule, and/or the capture or disruption of plaque or calcification within the vessel which may travel to other parts of the body.

Accordingly, there is a need for improved nosecones and delivery capsules that reduce the axial forces acting on the vasculature of the patient and the separation that forms between these components as a result.

Described herein are delivery apparatus and methods for implanting prosthetic heart valves, or other expandable medical devices. In particular, the disclosed delivery apparatus can comprise a nosecone configured to flexibly and resiliently curve or bend relative to an end portion thereof. A distal portion of the nosecone can comprise a material that is relatively softer and flexible than a proximal portion of the nosecone. A plurality of circumferential grooves that are coaxially aligned along the softer distal portion of the nosecone allows the nosecone to flex and bend as it contacts a surface external to the delivery apparatus. This, among other things, adds bendability and freedom of movement to the distal end of the delivery apparatus not found in typical delivery apparatus, to ease friction and limit gap formation caused by contact with the vessel wall. Further benefits can include a reduction in the frictional forces acting between the nosecone and the guidewire of the delivery apparatus as the nosecone moves axially along and with the bend of the guidewire.

In addition to the nosecone, the disclosed delivery apparatus can comprise a delivery capsule that comprises a tapered tip that extends at least partially over a flexible portion of the nosecone. The disclosed delivery capsule can comprise a main body configured to retain an expandable prosthetic device and receive an end portion of the nosecone. The tapered tip of the delivery capsule extends from a distal end of the main body and can be configured to extend over a seam formed by the distal most edge of the main body and the nosecone. The tapered tip can also extend at least partially over the tapered shape of a distal portion of the nosecone. The tapered tip can comprise a material that is relatively more flexible or pliable than a material that makes up the main body of the delivery capsule, which can, in some instances, stretch taut over the nosecone. This, among other things, allows the tapered tip to move with the nosecone as the nosecone bends and to form a seal around the seams of the delivery apparatus to eliminate or reduce fluid leakage into the delivery capsule or unwanted disruption of native tissue.

Additional information about the disclosed nosecones and delivery capsules, as well as exemplary delivery apparatus and prosthetic valves, is provided below.

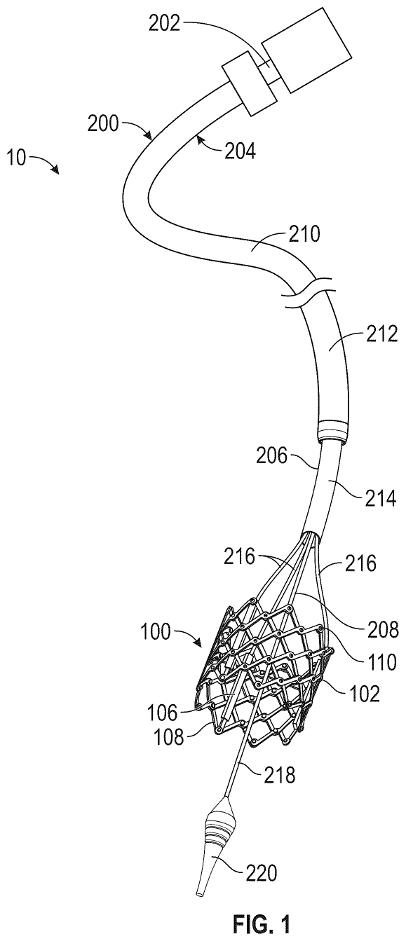

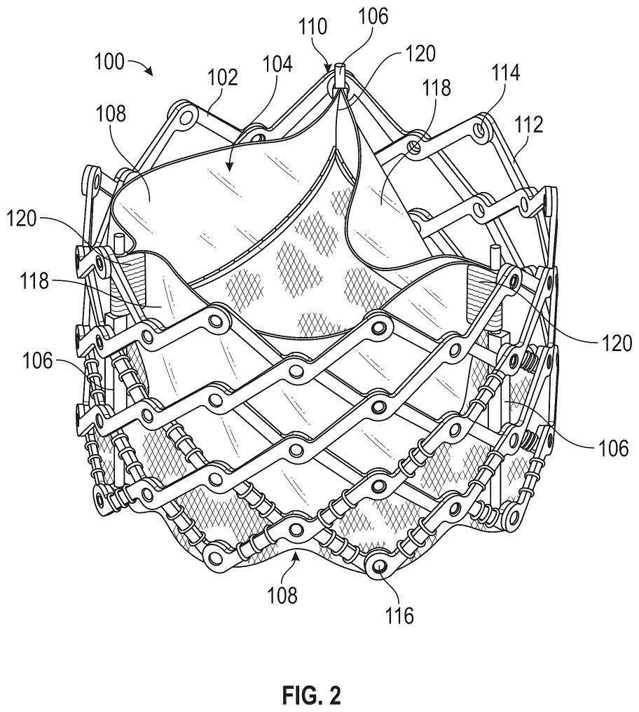

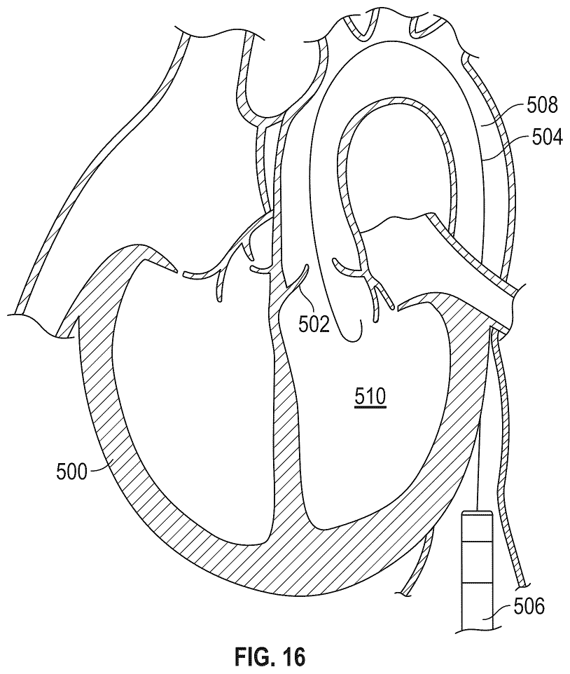

FIG. 1 depicts a delivery assembly 10, according to one example. In the illustrated example, the delivery assembly 10 comprises a prosthetic heart valve 100 and a delivery apparatus 200. The prosthetic heart valve 100 can be releasably coupled to the distal end portion of the delivery apparatus. The prosthetic heart valve 100 can be radially compressed to a delivery configuration and positioned within a delivery capsule of the delivery apparatus 200 (for example, FIG. 15). The delivery apparatus 200 can be used to insert the prosthetic heart valve 100 into a patient's vasculature and to position the prosthetic heart valve 100 relative to the patient's native anatomy. The delivery apparatus 200 can also be used to deploy the prosthetic heart valve 100 from the delivery capsule and (in some instances) to radially expand the prosthetic heart valve from the delivery configuration to a deployed, functional configuration (for example, FIGS. 2 and 22-23). An exemplary delivery procedure is described further below with reference to FIGS. 16-22. Additional details of the prosthetic heart valve 100 and the delivery apparatus 200 are also provided immediately below.

It should be noted at the outset that, although the exemplary prosthetic heart valves and delivery apparatus disclosed herein are primarily directed to transcatheter aortic valve implantation (TAVI), the technology and methods disclosed herein can be used and/or readily adapted for use in various other implantation locations and/or with various types of prosthetic devices. For example, the delivery apparatus disclosed herein can be configured for implanting a prosthetic valve at the native mitral, pulmonary, and/or tricuspid valve regions. Additionally, the delivery apparatus disclosed herein can be used with stents or other types of prosthetic devices that are disposed in a delivery capsule during a portion of an implantation procedure.

FIG. 2 depicts the prosthetic heart valve 100, which is an exemplary mechanically-expandable prosthetic heart valve. The prosthetic heart valve 100 comprises three main components: a frame 102, a valve structure 104, and a plurality of actuation members 106. In FIGS. 1, 14, and 19-22, the valve structure 104 is omitted to for ease of discussion and to better illustrate the frame 102 and the actuation members 106. The frame 102, which can also be referred to as “a stent” or “a support structure,” is configured for supporting the valve structure 104 and for securing the prosthetic heart valve 100 to native tissue (for example, a native heart valve annulus). Referring again to FIG. 2, the valve structure 104 can be coupled to the frame 102 and/or to the actuation members 106. The valve structure 104 is configured to allow blood flow through the prosthetic heart valve 100 in one direction (that is, antegrade) and to restrict blood flow through the prosthetic heart valve 100 in the opposite direction (that is, retrograde). In this manner, the prosthetic heart valve 100 comprise an inflow end 108 and an outflow end 110. The actuation members 106 are coupled to the frame 102 and are configured to adjust expansion of the frame 102 to a plurality of configurations including one or more functional or expanded configurations (for example, FIGS. 1 and 2), one or more delivery or compressed configurations (for example, FIG. 14), and/or one or more intermediate configurations between the functional and delivery configurations.

Still referring to FIG. 2, the frame 102 of the prosthetic heart valve 100 includes a plurality of interconnected struts 112 arranged in a lattice-type pattern. When the frame 102 is in a radially-expanded configuration, the struts 112 of the frame 102 extend diagonally relative to a longitudinal axis of the prosthetic heart valve 100. In other examples, the struts 112 of the frame 102 can be offset by a different amount than the amount depicted in FIG. 2. For example, FIG. 14 depicts the frame 102 in a radially-compressed configuration (which is also referred to herein as “a delivery configuration”). In the delivery configuration, the struts 112 of the frame 102 extend parallel (or at least substantially parallel) to the longitudinal axis of the prosthetic heart valve 100.

To facilitate movement between the expanded and compressed configurations, the struts 112 of the frame 102 are pivotably coupled to one another at one or more pivot joints 114. For example, the struts can comprise openings that are configured to receive pivot elements 116 (for example, rivets, pins, tabs, etc.). In some examples, each of the two pivotably-connected struts can comprise an opening, and the pivot element can extend through the opening of both struts. In other examples, a first strut of two pivotably-connected struts can comprise the pivot element (for example, fixedly attached thereto or integrally formed thereon), and a second strut of the two pivotably-connected struts strut can comprise an opening configured to receive the pivot element of the first strut. In any event, the pivot joints 114 allow the struts 112 to pivot relative to one another as the frame 102 moves between the radially-expanded configuration and the radially-compressed configuration.

The frame 102 of the prosthetic heart valve 100 can be made of any suitable materials, including biocompatible metals and/or biocompatible polymers. Exemplary biocompatible metals from which the frame can be formed include stainless steel, cobalt chromium alloy, and/or nickel titanium alloy (which can also be referred to as “NiTi” or “nitinol”).

With reference to FIG. 2, the valve structure 104 of the prosthetic heart valve 100 can comprise a plurality of leaflets 118 that collectively form a leaflet assembly. The leaflets 118 can be arranged to form commissures 120 (for example, pairs of adjacent leaflets), which can, for example, be mounted to respective actuation members 106 and/or to the frame 102.

The leaflets 118 of the prosthetic heart valve 100 can be made of a flexible material such that the leaflets 118 can open and close to regulate the one-way flow of blood through the valve structure 104. For example, the leaflets 118 can be made from in whole or in part, biological material, bio-compatible synthetic materials, and/or other such materials. Suitable biological material can include, for example, bovine pericardium, porcine pericardium, equine pericardium, ovine pericardium, etc.

Further details regarding prosthetic heart valves, including the manner in which the valve structure 104 can be coupled to the frame 102 of the prosthetic heart valve 100 and various actuators for expanding the prosthetic valve, can be found in U.S. Pat. Nos. 6,730,118, 7,393,360, 7,510,575, 7,993,394, and 8,652,202, U.S. Publication Nos. 2018/0153689 and 2018/0325665, and PCT Application No. PCT/US2020/057691, which are incorporated by reference herein.

As depicted in FIG. 2, the actuation members 106 of the prosthetic heart valve 100 are mounted to and spaced circumferentially around the interior of the frame 102. In the illustrated example, the prosthetic heart valve 100 comprises three actuation members 106. It should be noted that in other examples the prosthetic heart valve 100 can comprise fewer (for example, 1-2) or more (for example, 4-15) than three actuation members.

The actuation members 106 are configured to, among other things, radially expand and/or radially compress the frame 102. For this reason, the actuation members 106 can be referred to as “expansion mechanisms.” In some examples, the actuation members 106 can also be configured to lock the frame 102 at a desired expanded configuration. Accordingly, the actuation members 106 can also be referred to as “lockers” or “locking mechanisms.”

The actuation members 106 can be configured to form a releasable connection with one or more respective actuation shafts of a delivery apparatus (for example, FIG. 1). This can be accomplished in various ways, such as a threaded connection, a male/female mating connection, and/or various other means for releasably connecting.

FIG. 1 schematically depicts the delivery apparatus 200. The delivery apparatus 200 comprises a handle 202, a delivery catheter 204, an implant catheter 206, and a guidewire catheter 208. The implant catheter 206 extends axially through the delivery catheter 204, and the guidewire catheter 208 extends axially through the implant catheter 206 (and the delivery catheter 204). Each of the catheters 204, 206, 208 is movable relative to each other (for example, axially and/or rotationally). As schematically depicted, the proximal portions of the catheters 204, 206, 208 are coupled to the handle 202. In some examples, the delivery apparatus can comprise a plurality of handles, and the proximal portion of each catheter can be coupled to a respective handle.

Generally speaking, the delivery catheter 204 is configured to cover the prosthetic heart valve as the delivery assembly (that is, the delivery apparatus and the prosthetic heart valve) is inserted into a patient's vasculature and advanced to an implantation location. The implant catheter 206 is configured to be releasably coupled the prosthetic heart valve and to manipulate the expansion and/or contraction of the prosthetic heart valve at the implantation location. The guidewire catheter 208 is configured to track over a guidewire (which is inserted prior to insertion of the delivery apparatus 200) and route the delivery apparatus 200 to the implantation location.

Additional details about handles, delivery catheters, implant catheters, the guidewire catheters, releasably coupling the prosthetic heart valve to the delivery apparatus, and/or using the delivery apparatus to manipulate the prosthetic heart valve can be found, for example, in U.S. Publication Nos. 2018/0153689 and 2018/0311039 and U.S. Provisional Patent Application Nos. 62/990,299 and 62/945,039, which are incorporated by reference wherein.

Still referring to FIG. 1, the delivery catheter 204 comprises an outer shaft 210 and a delivery capsule 212 coupled to the distal portion of the outer shaft 210. In some examples, the outer shaft 210 and the delivery capsule 212 can be integrally formed (for example, co-molded) as a single, unitary component. In some examples, the outer shaft 210 and the delivery capsule 212 can be formed as separate components that are coupled together (for example, over-molding, bonding, adhesive, fasteners, and/or other means for coupling). Additional details about the delivery capsule 212 are provided below.

The implant catheter 206 comprises a main shaft 214 and one or more actuation shafts 216 extending through the main shaft 214. The actuation shafts 216 can be releasably coupled to the actuation members 106 of the prosthetic heart valve 100 and can be used to manipulate the prosthetic heart valve 100. The guidewire catheter 208 comprises a guidewire shaft 218 and a nosecone 220 coupled to the distal portion of the guidewire shaft 218. In some examples, the guidewire shaft 218 and at least a portion of the nosecone 220 (for example, a proximal portion) can be integrally formed as a single, unitary component. In some examples, the guidewire shaft 218 and the nosecone 220 can be formed as separate components that are coupled together (for example, over-molding, bonding, adhesive, fasteners, and/or other means for coupling). Additional details about the nosecone 220 are provided below.

In a typical delivery apparatus, the nosecone and delivery capsule form a relatively inflexibly rigid distal end that is advanced through a patient's vasculature during implantation. The patient's vasculature can include various curves, such as the relatively sharp curve of the aortic arch. When advanced through an aortic arch for example, a conventional nosecone may be pushed axially against the aortic wall as the rigidity and length of the combination of nosecone, delivery capsule, and prosthetic heart valve can make navigating the native arch challenging. This axial force may result in unwanted disruption of fat and calcium deposits along the vessel wall and gap formation between the nosecone and the distal end of the delivery capsule as the nosecone twists and/or rotates against the inner surface of the delivery capsule. Unwanted disruption of deposits and gap formation can, for example, result in leakage into the delivery capsule and loose deposits traveling within the body.

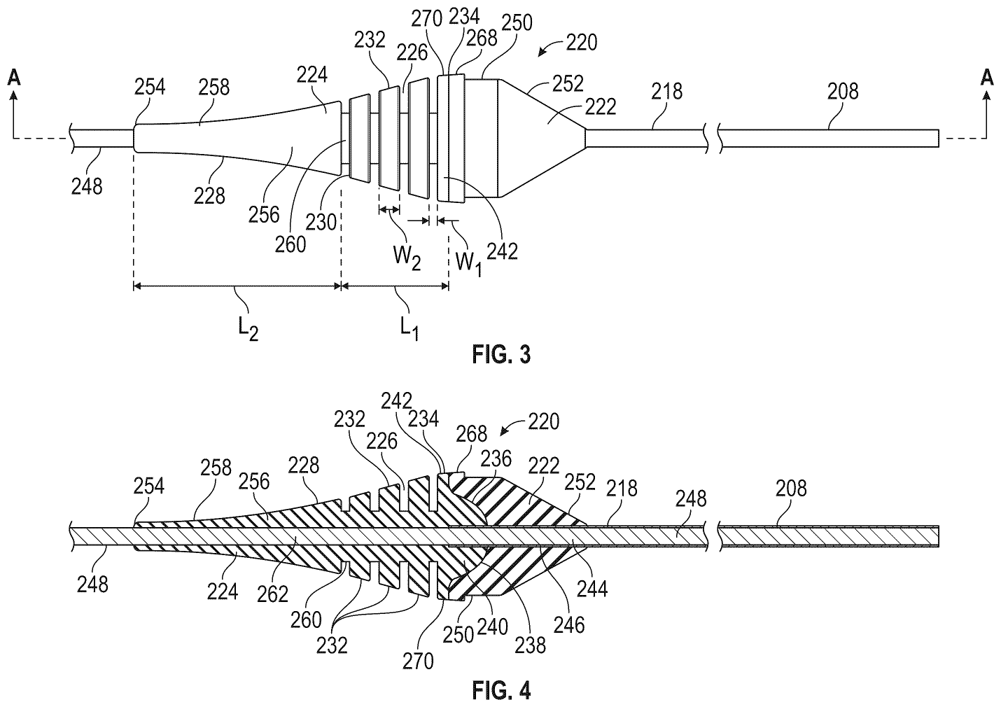

Described herein is a nosecone and delivery capsule (see, for example, FIGS. 3-4) configured to reduce the axial forces and/or gap formation created as the delivery assembly is advanced through a patient's vasculature. Generally speaking, the disclosed nosecone can, for example, provide added flexibility and movability to the distal end of the delivery apparatus to help ease friction and limit gap formation during vasculature procedures. In addition, the disclosed delivery capsule can effectively form a seal that significantly reduces unwanted leakage and/or capture of deposits in the event separation between the nosecone and the delivery capsule does occur.

Referring now to FIGS. 3 and 4, a nosecone 220 of the delivery apparatus according to one example comprises a proximal portion 222, a distal portion 224, and a plurality of grooves 226 along a length L1 of the distal portion 224. As shown in FIG. 3, the grooves 226 are formed of a series of discontinuous sections along the surface 228 of the distal portion 224. Each groove can have a first width W1 which spans the distance between a pair of opposing faces 230 that form the inner walls of each groove 226 and extend circumferentially around a circumference of the distal portion 224.

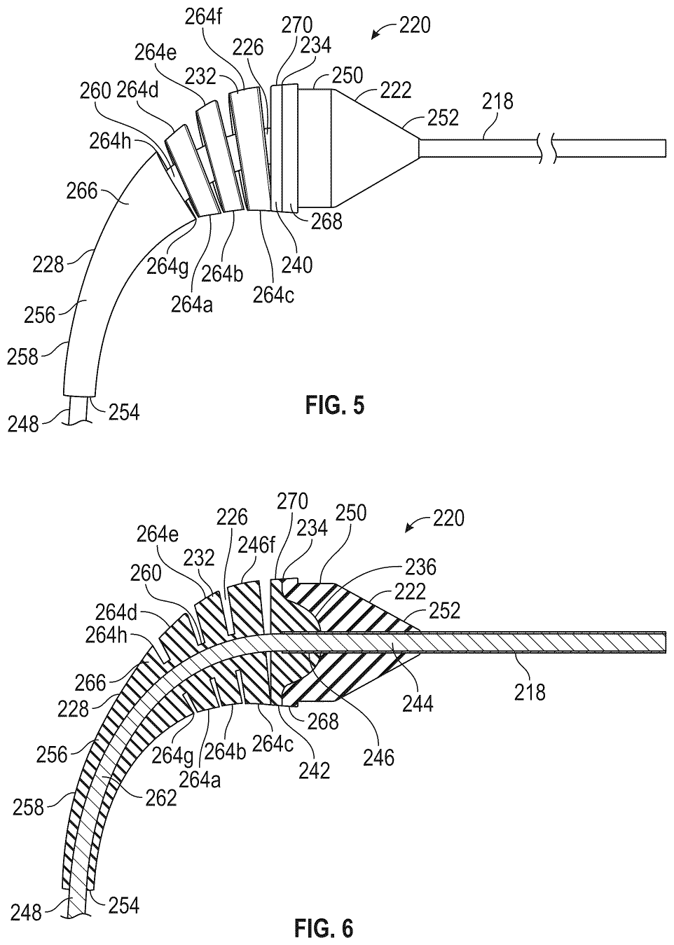

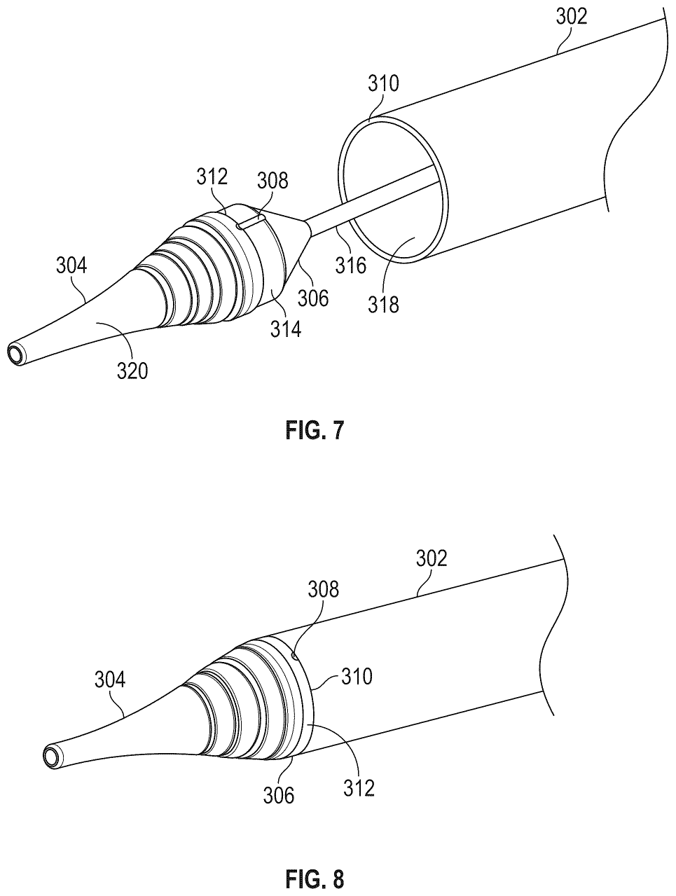

The grooves 226 can be axially spaced apart from one another along the length L1 of the distal portion 224 such that each pair of adjacent grooves 226 forms a rib 232 therebetween. Each rib 232 consequently can have a second width W2 equal to the axial spacing between a pair of corresponding adjacent grooves 226. Accordingly, the distal portion 224 can also be said to comprise a plurality of circumferential ribs 232 along a length L1 of the distal portion 224 with gaps extending therebetween. As will be described in further detail, the grooves 226 of the distal portion 224 are configured to allow each of the ribs 232 to move within the space or gap created by the grooves 226 such that the distal portion can bend relative to the proximal portion when the nosecone is manipulated between a straight state or configuration (for example, FIGS. 3-4) and a curved state or configuration (for example, FIGS. 5-6).

As shown in FIG. 3, the distal portion 224 of the illustrated example comprises four grooves 226 and three ribs 232 along the length L1 of the distal portion 224. Yet, in some examples, the distal portion 224 can include a greater or fewer number of grooves and ribs along a length of the distal portion 224, a length which can be greater than, less than, or equal to the length L1 depicted. For example, in some examples, the distal portion 224 can have a single groove 226 thereby bisecting the distal portion into two portions that allow the nosecone to bend. In other examples, the distal portion 224 can have two, three, five, or more grooves 226 and a corresponding number of ribs 232.

Still referring to FIGS. 3 and 4, the proximal portion 222 can be formed of a material having different material properties than a material that forms the distal portion 224. For instance, the proximal portion 222 can comprise a first material that is relatively harder or more ridged than a second material of the distal portion 224. By extension, the distal portion 224 can comprise a second material that is softer or more flexible relative to the first material of the proximal portion 222. Forming the distal portion 224 from a relatively softer or more flexible material can, for example, provide further flexibility to the nosecone 220 in addition to the grooves 226 and ribs 232. The relative softness or hardness of the materials of the nosecone can correspond with respective Shore durometer (D) values determined according to the appropriate methods and tools (for example, a durometer), however, any other metrics, methods, and/or tools may be used.

As illustrated in FIG. 4, which shows a cross-sectional profile of the nosecone 220 taken in a plane parallel to a longitudinal axis A of the nosecone 220, the proximal portion 222 and the distal portion 224 of the nosecone 220 can be coupled via a joint 236. The joint 236 can be situated at a midsection 234 of the nosecone 220 and comprise a cavity 238 sized and shaped to receive a similar sized and shaped projection 240 outwardly extending from a corresponding end of one of the proximal and distal portions. In the illustrated example of FIG. 4, for instance, the proximal portion 222 comprises a rounded cavity 238 configured to receive a rounded projection 240 that extends outwardly from a first end 242 of the distal portion 224. In the same manner, the arrangement of the joint 236 in some examples can be reversed, where the proximal portion 222 comprises the projection 240 and the distal portion 224 comprises the cavity 238.

In some examples, the proximal portion 222 and distal portion 224 described herein can be formed via a two-shot molding process. In such examples, the materials forming the proximal and distal portions 222, 224 are coupled to one another by way of chemical bond such that the two end portions form a unitary nosecone 220. The coupling of the proximal portion 222 and distal portion 224 in this way can provide structural integrity to the nosecone to offset the axial and/or torsional forces acting on the relatively softer distal portion as the nosecone contacts a vessel wall, thereby resisting separation between the proximal and distal end portions.

Although the joint 236 illustrated in FIG. 4 is a projection received within a cavity, other joints can be utilized in other examples, including a ball-and-socket joint, universal joint, hinge joint, saddle joint, or the like. In still further examples, the proximal portion 222 and distal portion 224 can be constructed of two or more separate components coupled and assembled by way of overmolding, through an adhesive, or a combination thereof.

Still referring to FIGS. 3 and 4, the illustrated example shows the proximal portion 222 can be coupled to the guidewire shaft 218 of the delivery apparatus 200. The proximal portion 222 can include a first lumen 244 that receives a distal end portion 246 of the guidewire shaft 218, which itself is sized to allow a guidewire 248 to pass therethrough. Because the proximal portion 222 can be formed of a relatively hard material, a strong coupling of the nosecone 220 to the guidewire shaft 218 can be obtained when, for example, overmolding is used to fix the proximal portion 222 to the guidewire shaft 218. Nonetheless, the proximal portion 222 can be coupled to the guidewire shaft 218 by a variety of methods, including radio-frequency welding, through an adhesive, or a combination thereof.

The proximal portion 222 can have both a cylindrical section 250 and a tapered section 252 that narrows toward the guidewire shaft 218. While a softer material of the distal portion 224 can provide added flexibility and mobility, the harder, more rigid material and tapered shape of the proximal portion 222 can reduce unwanted sticking between the nosecone 220 and the other components of the delivery assembly 10 when the delivery apparatus 200 is retrieved. For example, during retrieval of the delivery apparatus 200, the sloped profile of the proximal portion 222 directed toward the guidewire shaft 218 can help prevent the nosecone 220 from catching or sticking to the delivery capsule 212, the distal edge of the delivery catheter 204 (for example, an introducer sheath), and the prosthetic heart valve 100. Preventing sticking between the nosecone and heart valve can, for instance, prevent unwanted migration of the valve within the native annulus, which may occur if catching or sticking does take place.

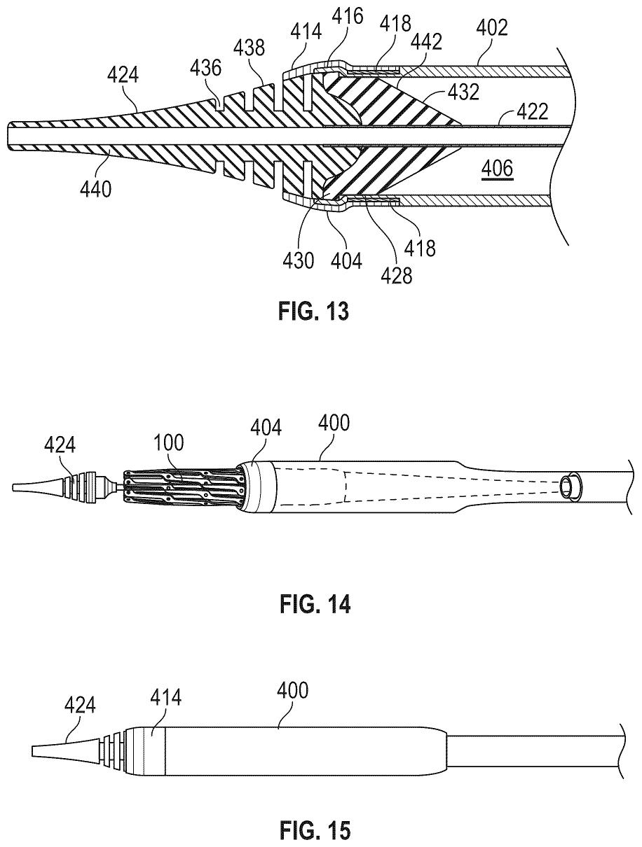

Another advantage of the nosecones described herein is the shortened length of the proximal portion situated within the delivery capsule during delivery of the prosthetic valve (for example, see FIG. 13). The length of a corresponding portion of a typical nosecone for instance, must be relatively long in comparison to the proximal portion of the nosecones described herein. In particular, the length of a typical nosecone within a capsule must be comparatively long in order to reduce separation between the nosecone and delivery capsule, separation which can occur via the force the typical nosecone exerts on the inner wall of the delivery capsule during delivery as axial forces act on the nosecone. By configuring the distal portion 224 of the nosecone 220 to bend relative the proximal portion 222, the length of the proximal portion 222 can thereby be shortened due to the reduction in force acting on the proximal portion 222 and inner wall of the delivery capsule 212. In some instances, the shortened length of the proximal portion 222 can also result in a relatively shorter delivery capsule, which no longer has to accommodate the relatively long length of a typical nosecone.

Referring again to FIGS. 3 and 4, the distal portion 224 of the nosecone 220 can have an overall tapered shape. The distal portion 224 can taper from a first end 242, proximate and in contact with the proximal portion 222, to a second end 254 located at the distal most end of the nosecone 220. Due to the tapered shaped of the distal portion 224, each rib 232 of the distal portion 224 can have a diameter less than the adjacent rib immediately preceding it, that is, the diameter of each successive rib 232 decreases as the distal portion 224 tapers from the first end 242 to the second end 254. By extension, the diameter of each rib 232 increases from the second end 254 to the first end 242.

The distal portion 224 also comprises a conical portion 256 that forms the narrow, tip portion of the distal portion 224, which is distal to the grooves 226 and ribs 232. The conical portion 256 itself tapers toward the second end 254 of the distal portion 224 and can have a length L2 which can be greater than, lesser than, or equal to the length L1 covered by the plurality of grooves 226 and ribs 232. The conical portion 256 can, for example, serve as the leading edge of the nosecone 220 and delivery apparatus 200 which comes in contact with an external surface, such as an inner vessel wall of the patient. For this purpose, the conical portion 256 can have a continuous outer surface 258 that allows the conical portion 256 to move more readily along the vessel wall during contact and to direct the distal end of the delivery apparatus 200 in an atraumatic fashion.

The distal portion 224 also comprises a central axial portion 260 that extends axially between the conical portion 256 and the first end 242. The central axial portion 260, and the material made therefrom, operates in conjunction with the arrangement of the ribs 232 and grooves 226 to allow the distal portion 224 to bend relative to the proximal portion 222. As illustrated in FIG. 3, the central axial portion 260 is formed by the grooves 226 extending circumferentially around and through the body of the distal portion 224. In this example, the circumferential ribs 232 can be said to be coaxially aligned along and extend from the central axial portion 260. For example, while the nosecone 220 is in a straight state (for example, FIGS. 3-4), the central axial portion 260 and the ribs 232 are aligned coaxially along a longitudinal axis A of the nosecone 220. Similarly, while the nosecone 220 is in its curved state (for example, FIGS. 5-6), the central axial portion 260 and ribs 232 are axially aligned along the curved axis of the distal portion 224 (for example, reflected by the curvature of a second lumen 262 and/or the guidewire 248).

The distal portion 224 can also comprise a second lumen 262 sized to receive and allow the guidewire 248 to pass therethrough. The second lumen 262 is aligned with and is an extension of the first lumen 244 of the proximal portion 222. The second lumen 262 can also be aligned coaxially with the central axial portion 260, ribs 232, and conical portion 256 of the distal portion 224 while the nosecone 220 is in its straight or curved state. Because the distal portion 224 as a whole is configured to bend relative to the proximal portion 222 of the nosecone 220, the second lumen 262 is formed within the softer, flexible body of the distal portion 224. In doing so, the second lumen 262 is configured to move along the curvature of the guidewire 248 as the delivery apparatus 200 is advanced over the guidewire 248 during implantation (for example, FIGS. 5-6). Contact between the inner wall of the second lumen 262 and the guidewire 248 may also influence or cause the guidewire 248 to curve as the nosecone 220 makes contact with a vessel wall and as the distal portion 224 bends.

As shown in the illustrated example of FIG. 4, the second lumen 262 of the distal portion 224 can also be sized to receive the distal end portion 246 of the guidewire shaft 218. For instance, a segment of the distal end portion 246 of the guidewire shaft 218 can extend into the second lumen 262. In this way, in some examples, the distal portion 224 can be coupled to the guidewire shaft 218 at the interface of the inner wall of the second lumen 262 and the distal end portion 246 of the guidewire shaft 218. Coupling of the distal portion 224 and the guidewire shaft 218 can be achieved via a two-shot molding process, overmolding, through an adhesive, or any combination thereof. In some examples, however, the distal end portion 246 of the guidewire shaft 218 does not extend into the second lumen 262, but only the first lumen 244.

As discussed above and depicted in FIGS. 5-6, the distal portion 224 of the nosecone 220 can bend relative to the proximal portion. In particular, if the nosecone 220 contacts the vessel wall as the delivery apparatus 200 (or delivery assembly 10) is advanced or retrieved, the structure of the nosecone allows the distal portion 224 to bend under the axial forces that result from contact with the vessel wall. As the distal portion 224 curves, diametrically opposing ends of each of the ribs move toward and away from one or more adjacent rib ends. For instance, when the distal portion 224 curves, rib ends 264a-c move within the space provided by the grooves and in the direction of an adjacent rib and the proximal portion 222. Specifically, rib end 264a is directed toward rib end 264b, rib end 264b is directed toward rib end 264c, and rib end 264c is directed to the first end 242 of the distal portion 224. Inversely, each of the rib ends 264d-f that diametrically oppose respective ribs ends 264a-c, curve radially and away from one another as the distal portion 224 curves. Similar to the diametrically opposing rib ends 264a-f, diametrically opposing ends 264g-h at a proximal end 266 of the conical portion 256 are directed toward rib end 264a and away from rib end 264d, respectively, as the distal portion curves.

Since the grooves 226 extend circumferentially around a circumference of the distal portion 224, the distal portion 224 can curve in any direction relative to longitudinal axis A (FIG. 3) and move about the longitudinal axis A of the nosecone 220 while in a curved state. The curvature of the distal portion 224, for instance, is not limited to the plane of curvature depicted in FIGS. 5-6, but can curve in any plane perpendicular to the longitudinal axis A. Accordingly, the conical portion 256, when observed from the distal end and directly down the longitudinal axis A of the nosecone 220, can move clockwise and counterclockwise around the longitudinal axis A while in a curved state.

As depicted in FIGS. 5-6, the relatively soft and flexible material forming the distal portion 224 allows the conical portion 256 to flex relative to the segment of the nosecone 220 that includes the grooves 226 and ribs 232. In particular, the conical portion 256 can flex from its proximal end 266 to the second end 254 of the nosecone 220 (that is, the distal most end of the conical portion 256). In some examples, the space between the ribs 232 provided by the grooves 226 permit the segment of the distal portion 224 with the grooves and ribs to flex more sharply than the conical portion 256 that has a continuous outer surface 258. In this way, the conical portion 256 can be said to be less flexible than the portion of the nosecone 220 that includes the grooves 226 and ribs 232. In other examples, the conical portion 256 can be equally or more flexible than the distal portion 224 that includes the grooves 226 and ribs 232. In still further examples, the conical portion 256 can comprise one or more additional grooves and corresponding number of ribs along its length or a portion thereof (for example, length L2) to provide further flexibility to the conical portion 256 and the distal portion 224 overall.

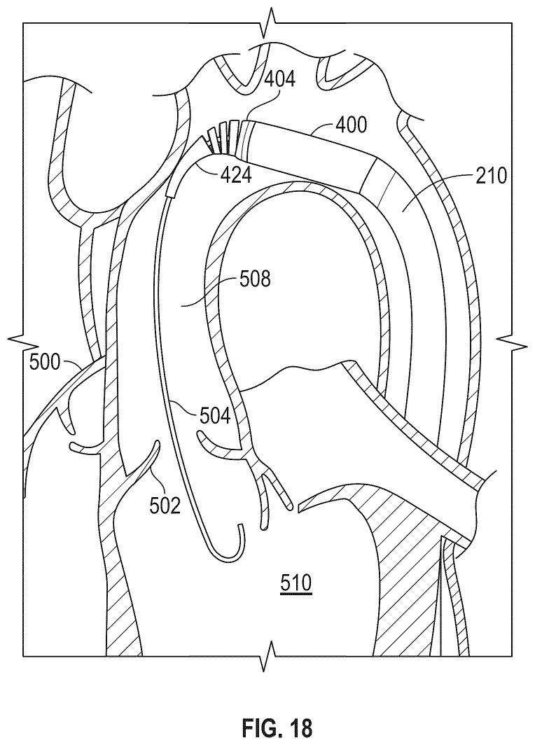

As described above and as illustrated in FIG. 6, the second lumen 262 extends through the central axial portion 260 and the conical portion 256 of the distal portion 224. Due to the relatively soft material of the distal portion 224, the distal portion 224 is configured to conform to the curvature of the guidewire 248 as the nosecone 220 is advanced over the guidewire 248 (see, FIGS. 17-18). Similarly, the inner wall of the second lumen 262 may contact and influence the curvature of the guidewire 248 as the nosecone 220 flexes upon contact with a vessel wall such that the curvature of the guidewire 248 conforms to the curvature of the second lumen 262 as the nosecone is advanced.

As shown in FIGS. 3-6, the proximal portion 222 and the distal portion 224 each comprise an annular outer ridge that extends outwardly from and circumferentially around their respective surfaces and are in contact with one another at the midsection 234 of the nosecone. An outer ridge 268 of the proximal portion 222 can form a portion of the nosecone 220 that contacts and/or is covered by a distal edge of the delivery capsule and an outer ridge 270 of the distal portion 224 can form the first end 242 the distal portion 224.

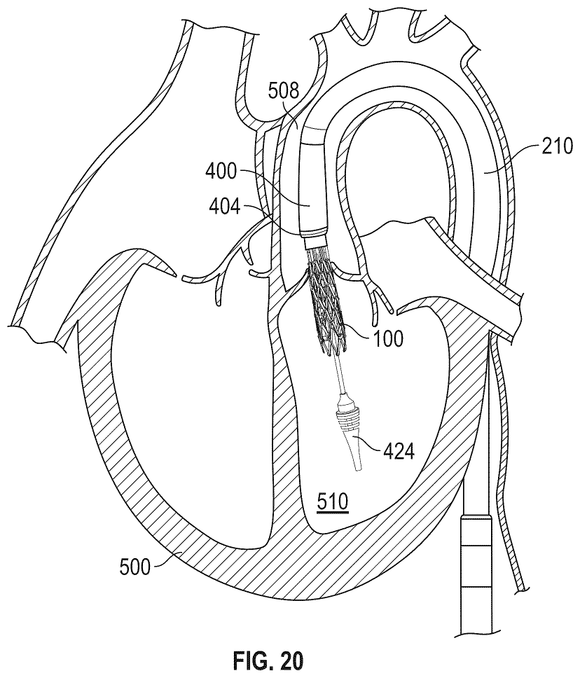

FIGS. 7-8 depict a distal end portion of the delivery apparatus 200 comprising a delivery capsule 302 and a nosecone 304 according to another example. The nosecone 304 comprises a proximal portion 306 coupled to a distal portion 320. The nosecone 304 is generally configured similar to the nosecone 220 and can include all of the features described above for the nosecone 220. One difference between the nosecone 304 and the nosecone 220, however, is that the proximal portion 306 of the nosecone 304 comprises a flush port 308. The delivery capsule 302 can be configured to receive and/or retain a prosthetic heart valve in a radially-compressed configuration (for example, FIGS. 14 and 20) and extend over the guidewire shaft 316 coupled to the nosecone 304. As shown in FIG. 8, a lumen 318 of the delivery capsule 302 receives the proximal portion 306, and contact between the distal edge 310 and the outer ridge 312 of the nosecone 304 effectively forms a seal which prevents fluid from flowing into the capsule 302.

Though it is generally desirable to prevent leakage or fluid from flowing into the lumen 318 from outside of the delivery apparatus 200, it may be desirable to allow some volume of fluid to flow outwardly from the lumen 318. For instance, the flush port 308, which forms an elongate groove extending through and along the surface of the cylindrical section 314 and the outer ridge 312 of the proximal portion 306, can allow fluid to flow outwardly from the lumen 318 and between the outer ridge 312 and distal edge 310 of the capsule 302. When the capsule 302 is in the closed position (as shown in FIG. 8), a portion of the flush port 308 extends distally beyond the distal edge 310 of the capsule 302. This, among other things, can permit a pressurized fluid flushed through the delivery catheter 204 to exit through flush port 308 when the capsule 302 is in the closed position, which can eliminate air bubbles from the delivery apparatus when the delivery apparatus 200 is being prepared for implantation. Though a single flush port 308 is depicted in FIGS. 7-8, the nosecone 304 can comprise two or more flush ports (for example, 3, 4, 6, etc.) arranged in a variety of arrangements.

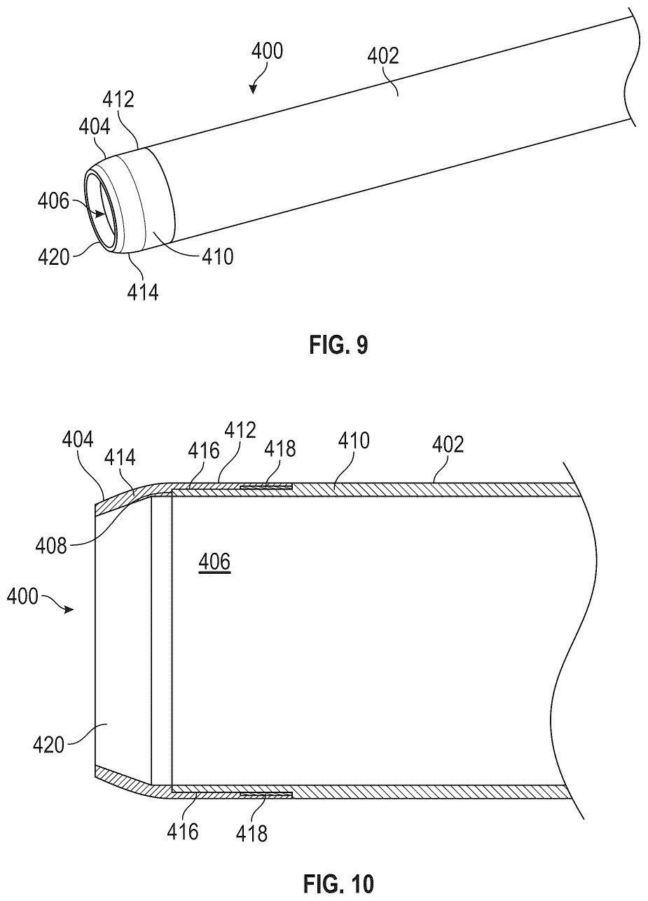

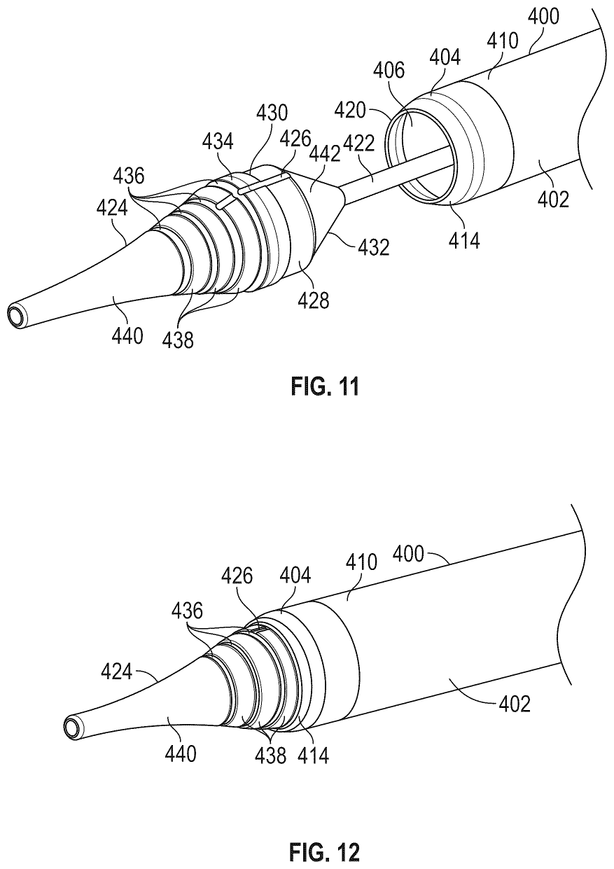

Now referring to FIGS. 9 and 10, a delivery capsule 400 of the delivery apparatus 200 according to one example comprises a main body 402 and a tapered-distal tip portion 404 extending axially from the main body 402. The main body 402 is configured to extend over a guidewire shaft 422 (FIGS. 11-13) and comprises a lumen 406 configured to receive and/or retain the prosthetic heart valve 100 in a radially-compressed configuration (FIGS. 14 and 20). The main body 402 also comprises a distal end portion 410 configured to extend over a proximal portion of a nosecone when the delivery capsule is in a closed position for delivery.

As shown in FIG. 10, the distal tip portion 404 (also referred to as a “distal rim”) comprises a tapered portion 414 and a cylindrical portion 412 coupled to the distal end portion 410 of the main body 402. The cylindrical portion 412 can, for instance, extend circumferentially around the distal end portion 410 and an annular distal edge 408 of the main body 402. In particular, as shown in FIG. 10, the outer surface of the distal end portion 410 can be formed with a recess in which the cylindrical portion 412 of the distal tip portion 404 is disposed. The outer diameter of the cylindrical portion 412 can be the same as the outer diameter of the distal end portion 410 such that the outer surfaces of the cylindrical portion 412 and the distal end portion 410 form a continuous outer cylindrical surface. Moreover, an inner surface of the cylindrical portion 412 and an outer surface of the distal end portion 410 can form an interface 416 and overlap in such a way that the inner surfaces of the tapered portion 414 and the distal end portion 410 form a continuous inner cylindrical surface within the lumen 406. In this way, a thickness of the delivery capsule 400 wall, that is, the thickness of the tapered portion 414, distal end portion 410, and overlapping segment of the cylindrical portion 412 and distal end portion 410, can be constant along the length of delivery capsule 400. In some examples, for instance, the thickness of the capsule wall can range from 0.25 mm to 0.35 mm. In some examples, the thickness of the capsule wall can be 0.30 mm. The cylindrical portion 412 of the distal tip portion 404 can be secured to the distal end portion 410 of the main body 402 in a variety of methods, such as by overmolding, an adhesive, welding, etc.

As shown in FIG. 10, a radiopaque marker band 418 can be embedded within the delivery capsule 400 and situated at the interface 416 formed by the inner surface of the cylindrical portion 412 and outer surface of the distal end portion 410. The marker band 418 in this way can increase visibility of the distal end portion of a delivery apparatus under fluoroscopy while inserted in the patient. The marker band 418 can also be constructed of a rigid or semi-rigid material, such as a nylon, Pebax®, polyurethane material, or a biocompatible metal, and have an inner diameter which allows the marker band 418 to extend over at least a portion of a proximal portion of a nosecone, but not over the entirety of the proximal portion. As one example, the marker band 418 can have an inner diameter greater than cylindrical and tapered sections of the proximal portion (for example, cylindrical and tapered sections 250, 252), but less than a diameter of an annular outer ridge (for example, outer ridge 268) of the proximal portion. In this instance, the marker band 418 forms a stop which limits axial movement of the delivery capsule 400 relative to the nosecone beyond a certain point (FIG. 13). In particular, as the distal tip portion 404 is directed axially toward the distal end of the nosecone (for example, while moving the capsule 400 into a closed position for delivery), relative axial movement between the delivery capsule 400 and the nosecone is prevented beyond the point at which the outer ridge, the marker band 418, and the material of the distal end portion 410 between the marker band 418 and outer ridge, meet. In this example, the delivery capsule 400 is prevented from moving beyond an undesired length of the nosecone, and the delivery capsule 400 and nosecone can be positioned securely against one another in a closed position during delivery to seal the lumen 406 of the capsule.

FIGS. 9 and 10 show that the distal tip portion 404 extends axially from the distal end portion 410 and forms an opening 420 to the delivery capsule 400. The tapered portion 414 of the distal tip portion 404 can be configured, for example, to extend at least partially over an outer surface of a nosecone of the delivery apparatus. Specifically, the distal tip portion 404 of the delivery capsule can extend over and conform to the shape of a corresponding tapered section and outer ridges of the nosecones described herein. As an example, when the delivery capsule 400 is in a closed configuration for delivery, the tapered portion 414 can extend partially over the tapered distal portion (for example, distal portion 320) and fully over the outer ridges (for example, outer ridge 312) of the nosecone when the marker band 418, outer ridge of the nosecone, and the material of the distal end portion 410 are sandwiched against one another. This, among other things, allows the distal tip to help further prevent fluid leakage into the delivery capsule and unwanted disruption of the vessel wall in the event separation forms between the nosecone and the delivery capsule 400.

Similar to the nosecone 220, the distal tip portion 404 can be formed of a material having different material properties than a material that forms the main body 402. For instance, the distal tip portion 404 can comprise a first material that is relatively softer or more flexible than a second material of the main body 402. By extension, the main body 402 can comprise a second material that is harder or more rigid relative to the first material of the distal tip 404. This, among other things, allows the distal tip portion 404 to fit securely around a nosecone without hindering movement of the nosecone. The relative hardness or softness of the materials of the delivery capsule can, for example, correspond with respective Shore D values, however, any other metrics may be used. In some examples, the distal tip portion 404 can be formed from a polyether block amide (PEBA) thermoplastic elastomer and the delivery capsule 400 can be formed of a nylon polymer that is relatively harder than the PEBA polymer of the distal tip portion 404. In some examples, the distal tip portion 404 can be made of a PEBA elastomer (for example, Pebax®) and can have a durometer of 45 D or less, such as 35 D, or 30 D.

Turning to FIGS. 11-13, a distal end portion of the delivery apparatus 200 comprises the delivery capsule 400 and a nosecone 424 according to another example. The nosecone 424 is generally configured similar to the nosecone 304 and the nosecone 220 and can include all of the features described above for the nosecone 304 and the nosecone 220. One difference between the nosecone 424 and the nosecone 304 and the nosecone 220, however, is that a flush port 426 forms an elongate groove extending through and along the surface of both a proximal portion 432 and a distal portion 440 of the nosecone 424. Specifically, the flush port 426 extends through and along a cylindrical section 428 and an outer annular ridge 430 of the proximal portion 432 as well as through and along an annular outer ridge 434 and one or more grooves 436 and ribs 438 of the distal portion 440. Although only one flush port 426 is shown FIGS. 11-12, it should be appreciated that, in some examples, two or more flush ports can be included in a variety of arrangements.

As shown in FIGS. 12-13, the distal tip portion 404 of the delivery capsule 400 is configured to extend partially over a corresponding tapered section of the distal portion 440 (that is, partially over the decreasing circumference of one or more of the ribs 438) when the capsule 400 is in the closed position. In the illustrated example, for instance, the tapered portion 414 of the distal tip portion 404 extends over and conforms (for example, molds and stretches) to the shape of the outer ridge 434, a proximal-most groove 436, and a proximal-most rib 438 of the distal portion 440 when the capsule 400 is in the closed position.

The extension of the distal tip portion 404 over the distal portion 440 can, for example, help prevent fluid external to the delivery apparatus 200 from leaking between the interface between the capsule and the nosecone and into the lumen 406 during implantation. Also, since the distal tip portion 404 is in contact (or in near contact) with the distal portion 440 of the nosecone 424, unwanted disruption of deposits on the vessel wall may also be avoided because the distal tip 404 covers the separation between the capsule and nosecone that might otherwise cause disruption. While in the closed position, one or more flush ports 426 can be configured to allow a pressurized flush fluid to flow outwardly from the lumen 406 of the delivery capsule 400, between the distal edge of the distal tip portion 404 and the outer surface of the proximal portion 432 and distal portion 440 where the flush port 426 extends along. As illustrated in FIG. 12, a portion of the flush port 426 extends distally beyond the distal edge of the distal tip portion 404 along one or more ribs 438 when the capsule 400 is in the closed position.

In some examples, the distal tip 404 can be configured to extend over two or more grooves 436 and/or ribs 438. In such examples, one or more flush ports 426 can extend through a corresponding number of ribs 438 to permit fluid to flow therethrough and out of the lumen 406.

In some examples, the softer, more flexible material that forms the distal tip portion 404 is configured to elastically stretch taut and circumferentially around the tapered shape of the distal portion 440 of the nosecone 424. In other examples, the material that forms the distal tip portion 404 may be configured to be taut around the distal portion 440 and sufficiently rigid to permit the proximal portion 432 to be drawn back into the lumen 406 without buckling once the prosthetic heart valve 100 has been deployed. In other words, the distal tip 404 may be configured by way of its material to allow the nosecone to be received back within the delivery capsule 400 without a degree of material buckling or bunching that would otherwise prevent the nosecone from being retracted back into the lumen 406.

As mentioned and as shown in FIG. 13, the delivery capsule 400 can include a radiopaque marker band 418 situated at the interface 416 of the cylindrical portion 412 and distal end portion 410 of the delivery capsule 400. As depicted in FIG. 13, the marker band 418 can have an inner diameter which allows the marker band 418 and inner surface of the capsule 400 to extend over a cylindrical section 428 and a tapered section 442 of the proximal portion 432 of the nosecone 424. At the same time, the inner diameter of the marker band 418 can be less than the diameter of the annular ridge 430 of the proximal portion 432 such that the annular ridge 430 prevents further axial movement of the delivery capsule 400 beyond the point where the annular ridge 430, the marker band 418, and the material of the distal end portion 410 between the marker band 418 and outer ridge 430 come in contact. The inner diameter of the marker band 418 is therefore greater than a diameter of the lumen 406 (that is, inner surface) of the main body 402 of the capsule and less than a diameter of the annular ridge 430. In some examples, the annular ridge 430 of the proximal can be eliminated, in which case the marker band 418 and the material of the distal end portion 410 can contact the annular ridge 434 of the distal portion 440.

In some examples, the marker band 418 has an inner diameter such that the inner surface of the delivery capsule 400 fits tightly against the cylindrical section 428 of the proximal portion 432 of the nosecone 424, thereby creating a seal for the lumen 406 of the delivery capsule. In such examples, the inner surface of the delivery capsule 400 transitions between a first diameter equal or substantially equal to the diameter of the cylindrical section 428 and a second diameter equal or substantially equal to the diameter of the annular ridge 430. In some examples, the diameter of the cylindrical section 428 can range from 7.25 mm to 7.35 mm, while in other examples, the cylindrical section 428 can have a diameter of 7.3 mm. In still further examples, the diameter of the annular ridge 430 can range from 7.55 mm to 7.65 mm or range from 7.85 mm to 7.95 mm. In other examples, the annular ridge 430 can have a diameter of 7.60 mm or 7.90 mm. Although diameters of the cylindrical section 428 and annular ridge 430 are described with particularity, it should be appreciated the cylindrical section and annular ridge can have any diameter suitable for delivery within a native annulus.

FIGS. 14-15 depict the prosthetic heart valve 100 being loaded into the delivery capsule 400. More specifically, FIG. 14 depicts the prosthetic heart valve 100 partially loaded into the delivery capsule 400, and FIG. 15 depicts the prosthetics heart valve 100 fully loaded into the delivery capsule 400. In the illustrated example, the distal tip 404 extends partially over the nosecone 424.

FIGS. 16-22 schematically depict an exemplary implantation procedure in which a delivery assembly comprising the prosthetic heart valve 100 and the delivery apparatus 200 (with the delivery capsule 400 and nosecone 424 in lieu of nosecone 220) is used to implant the prosthetic heart valve 100 in a native aortic valve 502 of a heart 500 using a transfemoral delivery procedure.

As depicted in FIG. 16, a guidewire 504 is inserted into the patient's vasculature (via a surgical incision in a femoral artery) and extends through the patient's aorta 508 and into the patient's left ventricle 510 using a retrograde approach. An introducer device 506 can be inserted over the guidewire into the patient's vasculature. As depicted in FIG. 17, the distal end portion of the delivery assembly is advanced over the guidewire 504 and inserted into the patient's vasculature via the introducer device 506.

As shown in FIG. 18, as an axial force is applied to the delivery apparatus 200 (for example, at the handle 202) to advance the delivery apparatus 200 around the aortic arch 508, the nosecone 424 and delivery capsule 400 may contact the surrounding aortic wall. The softer, flexible distal portion of the nosecone 424 can bend relative to delivery capsule 400 thereby allowing the delivery apparatus to pass the through the aortic arch with relatively low axial forces compared to the forces required for typical nosecones and delivery capsule. The delivery capsule 400 can also cover any separation between the nosecone 424 and delivery capsule 400 in the event the separation forms, which can, for example, eliminate or reduce leakage and unwanted disruption of the vessel wall.

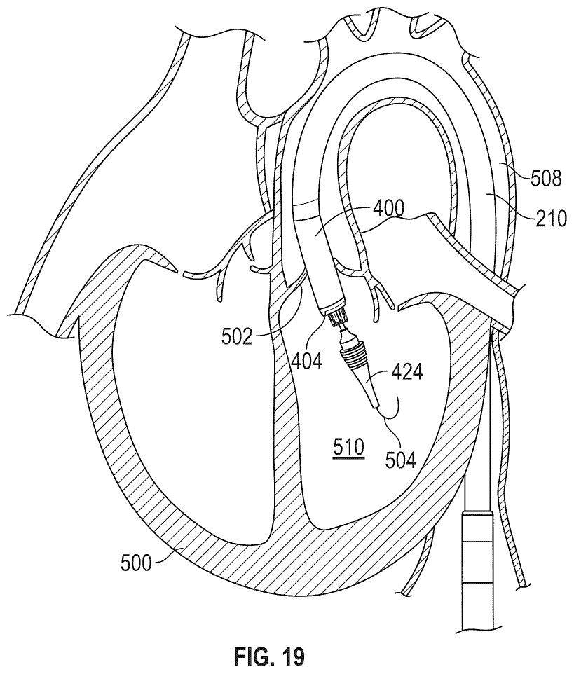

Referring now to FIG. 19, the distal end portion of the delivery assembly is positioned such that the delivery capsule is disposed within the native aortic valve 502. The prosthetic heart valve 100 can be positioned relative to the native anatomy. For example, the prosthetic heart valve can be positioned such that the coronary ostia are unobstructed (or less obstructed). This can be accomplished by positioning the delivery capsule 400 relative to the native anatomy. During the positioning of the delivery capsule 400 and the prosthetic heart valve 100, the prosthetic heart valve can be fully disposed within the delivery capsule (see, for example, FIG. 15) or partially disposed within the delivery capsule and partially exposed from the delivery capsule (see, for example, FIGS. 14 and 19).

Although FIGS. 19-20 depict the prosthetic heart valve 100 being deployed from the delivery capsule while the delivery capsule and prosthetic heart valve are disposed within the native aortic valve annulus, in other implementations, the delivery capsule can be disposed more superior (for example, toward the ascending aorta) or more inferior (for example, toward the left ventricle) during valve deployment.

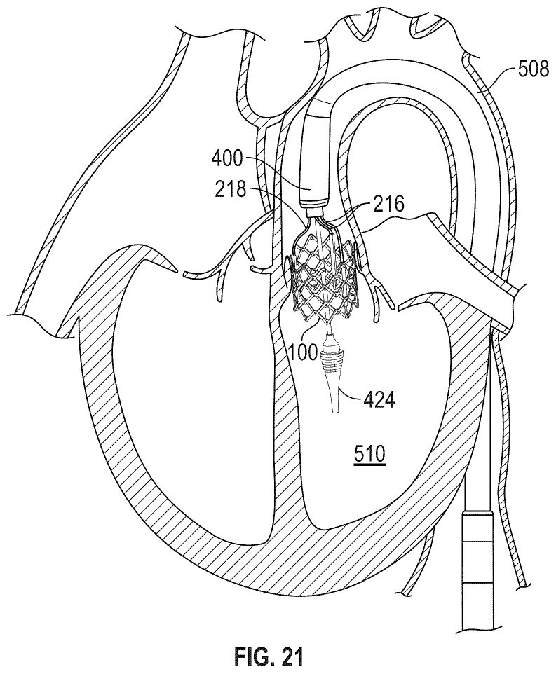

With the prosthetic heart valve 100 positioned as desired, the prosthetic heart valve 100 can be fully deployed from the delivery capsule 400 (see FIG. 20). The prosthetic heart valve 100 can then be expanded from the radially-compressed configuration to a radially-expanded configuration, as shown for example in FIG. 21. In the illustrated example, the prosthetic heart valve 100 is a mechanically-expandable prosthetic heart valve, which is expanded via the delivery apparatus actuating the actuators of the prosthetic heart valve. In other examples, the prosthetic heart valve can be a self-expandable prosthetic heart valve or a balloon-expandable prosthetic heart valve.

In some examples, the prosthetic heart valve can be expanded in a plurality of ways. For example, a prosthetic heart valve may be self-expanding (for example, due to super-elastic and/or shape-memory properties of the frame) from a delivery configuration to a first expanded configuration and mechanically-expanding (for example, via actuators) from the first expanded configuration to a second expanded configuration, which is radially larger than the first expanded configuration. As another example, a prosthetic heart valve may be self-expanding (for example, due to super-elastic and/or shape-memory properties of the frame) from the delivery configuration to the first expanded configuration and balloon-expandable from the first expanded configuration to the second expanded configuration, which is radially larger than the first expanded configuration. In some examples, a prosthetic valve can be fully self-expandable or fully balloon-expandable.

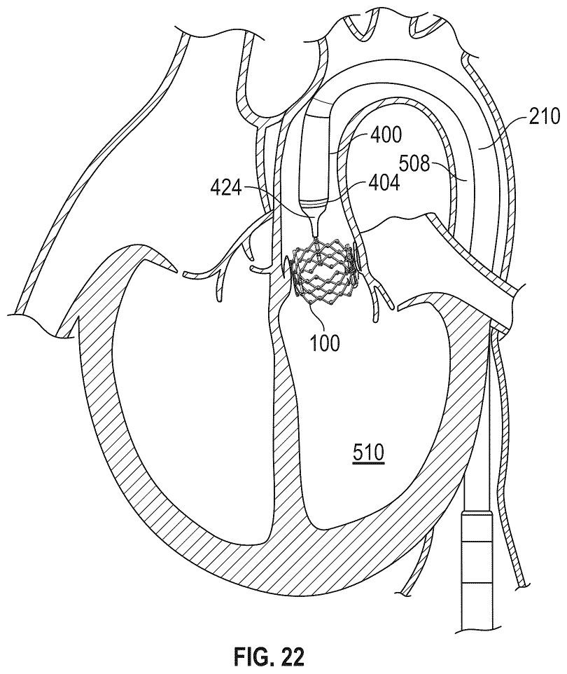

The fully expanded prosthetic heart valve 100 is secured relative to the native anatomy. As such, the prosthetic heart valve 100 can be released from the delivery apparatus 200, and the delivery apparatus 200 can be retracted from the patient's vasculature, as depicted in FIG. 22.

It should be noted that the nosecones and delivery capsules disclosed herein (for example, the nosecone 424 and delivery capsule 400) can be configured for use with various types of prosthetic heart valve and/or other types of prosthetic implants.

In some examples, the prosthetic heart valve 100 can be radially compressed (for example, via actuators and/or a crimping device) and loaded into the delivery capsule 400. In certain examples, the delivery capsule 400 is configured to receive both the prosthetic heart valve 100 and a portion of the nosecone 424.

In alternative examples, the nosecones and delivery capsules disclosed herein can be incorporated in delivery apparatuses that are configured to implant a plastically-expandable prosthetic valve or a self-expandable prosthetic valve without the use of actuators. In one implementation, a delivery assembly can comprise a plastically-expandable prosthetic valve and a delivery apparatus that includes an inflatable balloon for deploying the prosthetic valve. The prosthetic valve can comprise a radially expandable frame made of a plastically expandable material, such as stainless steel or a cobalt-chromium alloy (for example, MP35N®). Examples of plastically-expandable valves are disclosed in U.S. Pat. No. 9,393,110, and U.S. Publication Nos. 2018/0028310 and 2019/0365530, which are incorporated herein by reference. Examples of delivery apparatuses with inflatable balloons for implanting such prosthetic valves are disclosed in U.S. Publication Nos. 2013/0030519 and 2009/0281619, which are incorporated herein by reference. Such delivery apparatuses (and others with inflatable balloons) can incorporate any of the nosecones disclosed herein and/or any of the delivery capsules disclosed herein. In use, the prosthetic valve can be radially crimped on or adjacent the balloon (which is initially uninflated), delivered to a native heart valve (such as shown in FIGS. 17-19) and radially expanded at the desired implantation site by inflating the balloon.

In another implantation, a delivery assembly can comprise a self-expandable prosthetic valve and a delivery apparatus for implanting the prosthetic valve. The prosthetic valve can comprise a self-expandable frame made of a shape-memory material, such as Nitinol. Examples of self-expandable prosthetic valves are disclosed in U.S. Pat. No. 8,652,202 and U.S. Publication No. 2019/0262129, which are incorporated herein by reference. Examples of delivery apparatuses for implanting such prosthetic valves are disclosed in U.S. Pat. Nos. 8,652,202 and 9,155,619 and U.S. Publication Nos. 2014/0343670 and 2019/0008640, which are incorporated herein by reference. Such delivery apparatuses can incorporate any of the nosecones disclosed herein and/or any of the delivery capsules disclosed herein. In use, the prosthetic valve can loaded into a delivery capsule of a delivery apparatus and retained in a radially compressed state within the delivery capsule. The prosthetic valve can then delivered to a native heart valve (such as shown in FIGS. 17-19), and the advanced from the delivery capsule such that the prosthetic valve self-expands from the radially compressed state to a radially expanded state.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### ADDITIONAL EXAMPLES OF THE DISCLOSED TECHNOLOGY

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1: A delivery apparatus for an expandable prosthetic heart valve, the delivery apparatus comprising: a handle; a shaft having a proximal portion and a distal portion, the proximal portion being coupled to the handle; and a nosecone coupled to the distal portion of the shaft, the nosecone comprising a proximal portion and a distal portion coupled to the proximal portion, wherein the distal portion is configured to flexibly curve relative to the proximal portion and the proximal portion is formed of a first material that is relatively harder than a second material forming the distal portion.

Example 2: The delivery apparatus of any example herein, particularly example 1, wherein the distal portion of the nosecone comprises a plurality of axially spaced circumferential grooves.

Example 3: The delivery apparatus of any example herein, particularly example 2, wherein the plurality of circumferential grooves is formed along a tapered section of the distal portion of the nosecone.

Example 4: The delivery apparatus of any example herein, particularly any one of examples 2-3, wherein the plurality of circumferential grooves defines a plurality of ribs that move relative to each other when the distal portion flexibly curves relative to the proximal portion.

Example 5: The delivery apparatus of any example herein, particularly any one of examples 2-4, wherein the nosecone comprises a central axial portion extending axially between each circumferential groove of the distal portion.

Example 6: The delivery apparatus of any example herein, particularly any one of examples 1-5, wherein the proximal and distal portions of the nosecone are coupled by a projection received in a cavity, wherein one of the proximal portion and the distal portion comprises the projection and the other of the proximal portion and distal portion comprises the cavity.

Example 7: The delivery apparatus of any example herein, particularly example 6, wherein the distal portion comprises the projection and the proximal portion comprises the cavity.

Example 8: The delivery apparatus of any example herein, particularly example 6, wherein the proximal portion comprises the projection and the distal portion comprises the cavity.

Example 9: The delivery apparatus of any example herein, particularly any one of examples 6-8, wherein the projection comprises a rounded cross-sectional profile taken in a plane parallel to a longitudinal axis of the nosecone.

Example 10: The delivery apparatus of any example herein, particularly any one of examples 6-9, wherein the proximal portion comprises an outer surface, an outer ridge extending outwardly from and circumferentially around the outer surface, and one or more flush ports extending through and along the outer surface and outer ridge of the proximal portion.

Example 11: The delivery apparatus of any example herein, particularly any one of examples 1-10, wherein the distal portion comprises a first end proximate the proximal portion and a second end distal the first end, and wherein the distal portion tapers from the first end to the second end.

Example 12: The delivery apparatus of any example herein, particularly any one of examples 2-11, wherein the distal portion comprises a conical portion distal to the plurality of grooves, and wherein the conical portion is configured to bend relative to the proximal portion.

Example 13: The delivery apparatus of any example herein, particularly example 1, wherein the shaft comprises a first shaft, and the delivery apparatus further comprises a second shaft extending over the first shaft and a delivery capsule coupled to the second shaft, wherein the delivery capsule is configured to receive the proximal portion of the nosecone and retain an expandable prosthetic heart valve in a radially-compressed configuration for delivery into a patient.

Example 14: The delivery apparatus of any example herein, particularly example 13, wherein the delivery capsule is configured to extend over the proximal portion and partially over the distal portion of the nosecone.

Example 15: The delivery apparatus of any example herein, particularly any one of examples 13-14, wherein the delivery capsule comprises a tapered distal end portion configured to extend partially over the distal portion of the nosecone.

Example 16: The delivery apparatus of any example herein, particularly any one of examples 14-15, wherein the proximal portion of the nosecone comprises one or more flush ports extending along an outer surface of the proximal portion, and wherein the flush ports are configured to allow a fluid to flow therethrough when the delivery capsule is in a closed position extending over the proximal portion.

Example 17: The delivery apparatus of any example herein, particularly any one of examples 1-16, wherein the proximal portion of the nosecone comprises a first lumen and the distal portion of the nosecone comprises a second lumen, and wherein the first and second lumens are sized to receive a guidewire therethrough.

Example 18: The delivery apparatus of any example herein, particularly example 17, wherein the second lumen is configured to have the same curvature as the distal portion when the distal portion curves relative to the proximal portion of the nosecone.

Example 19: The delivery apparatus of any example herein, particularly any one of examples 17-18, wherein the distal portion of the shaft extends into the first lumen, and wherein the proximal portion of the nosecone is fixed to the distal portion of the shaft.

Example 20: A delivery apparatus for an expandable prosthetic heart valve, the delivery apparatus comprising: a handle; and a first shaft and a second shaft extending over the first shaft, each shaft comprising a distal portion and a proximal portion coupled to the handle, the distal portion of the second shaft being coupled to a delivery capsule of the delivery apparatus, wherein the delivery capsule comprises a main body configured to retain an expandable prosthetic heart valve in a radially-compressed configuration for delivery into a patient and a tapered-distal tip portion axially extending from the main body and tapering in a distal direction.

Example 21: The delivery apparatus of any example herein, particularly example 20, wherein a material forming the tapered-distal tip portion has a hardness less than a material forming the main body.

Example 22: The delivery apparatus of any example herein, particularly any one of examples 20-21, wherein the tapered-distal tip portion extends circumferentially around a distal portion of the main body.

Example 23: The delivery apparatus of any example herein, particularly any one of examples 20-22, wherein the tapered-distal tip portion comprises a cylindrical portion and a tapered portion, and wherein the cylindrical portion extends circumferentially around a distal end portion of the main body and the tapered-distal tip portion tapers in a direction extending distally from the cylindrical portion and forms an opening to the delivery capsule.

Example 24: The delivery apparatus of any example herein, particularly any one of examples 20-23, wherein an outer surface of the main body and an outer surface of the tapered-distal tip portion form a continuous outer surface of the delivery capsule.

Example 25: The delivery apparatus of any example herein, particularly any one of examples 20-24, wherein an inner surface of the main body and an inner surface of the tapered-distal tip portion form a continuous inner surface of the delivery capsule.

Example 26: The delivery apparatus of any example herein, particularly any one of examples 20-23, wherein the main body comprises an outer surface and the tapered-distal tip portion comprises an inner surface, and wherein the outer surface of the main body and the inner surface of the tapered-distal tip portion form an interface where the tapered-distal tip portion extends circumferentially over the main body.

Example 27: The delivery apparatus of any example herein, particularly example 26, wherein the delivery capsule comprises at least one radiopaque marker band located at the interface formed by the main body and the tapered-distal tip portion.

Example 28: The delivery apparatus of any example herein, particularly any one of examples 20-27, further comprising a nosecone coupled to the distal portion of the first shaft, wherein the delivery capsule is configured to move to a closed position in which the tapered-distal tip portion of the delivery capsule extends along an outer surface of the nosecone.

Example 29: The delivery apparatus of any example herein, particularly example 28, wherein the tapered-distal tip portion of the delivery capsule extends along a tapered portion of the nosecone when the delivery capsule is in the closed position.

Example 30: The delivery apparatus of any example herein, particularly any one of examples 27-28, wherein the tapered-distal tip portion extends over one or more portions of the nosecone.

Example 31: The delivery apparatus of any example herein, particularly any one of examples 20-30, wherein the delivery capsule is configured to receive a proximal portion of a nosecone.

Example 32: The delivery apparatus of any example herein, particularly any one of examples 28-31, wherein the nosecone comprises one or more flush ports extending along the outer surface of the nosecone, and wherein the one or more flush ports are configured to allow fluid to flow therethrough.

Example 33: The delivery apparatus of any example herein, particularly any one of examples 28-32, when depending from example 27, wherein the radiopaque marker band is configured to limit relative axial movement between the delivery capsule and the nosecone when the delivery capsule is in a closed position.

Example 34: The delivery apparatus of any example herein, particularly example 33, wherein the nosecone comprises an outer ridge and the radiopaque marker band has an inner diameter less than a diameter of the outer ridge.

Example 35: The delivery apparatus of any example herein, particularly any one of examples 33-34, wherein an inner surface of the delivery capsule transitions from a first diameter to a second diameter when the delivery capsule is in a closed position.

Example 36: The delivery apparatus of any example herein, particularly example 35, wherein the first diameter is less than or equal to the inner diameter of the radiopaque marker band and the second diameter is greater than or equal to the diameter of the outer ridge of the nosecone.

Example 37: The delivery apparatus of any example herein, particularly any one of examples 34-36, wherein a material of the delivery capsule is situated between the radiopaque marker band and the outer ridge of the nosecone when the delivery capsule is in a closed position.

Example 38: A delivery apparatus for an expandable prosthetic heart valve, the delivery apparatus comprising: a handle; a first shaft and a second shaft extending over the first shaft, each shaft comprising a distal portion and a proximal portion coupled to the handle, the distal portion of the second shaft coupled to a delivery capsule of the delivery apparatus, wherein the delivery capsule comprises a main body configured to retain an expandable prosthetic heart valve in a radially-compressed configuration for delivery into a patient and a distal rim axially extending from the main body; and a nosecone coupled to the distal portion of the first shaft, the nosecone comprising a proximal portion and a distal portion coupled to the proximal portion and configured to bend relative to the proximal portion and the delivery capsule; wherein the delivery capsule is movable between a closed position and an open position, wherein when delivery capsule is in the closed position, the main body of the delivery capsule receives the proximal portion of the nosecone, and the distal rim of delivery capsule extends over the proximal portion of the nosecone and partially over the distal portion of the nosecone, and wherein when the delivery capsule is in the open position, the distal rim is spaced proximally from the nosecone.

Example 39: The delivery apparatus of any example herein, particularly example 38, wherein the proximal portion is formed of a first material that is relatively harder than a second material forming the distal portion.

Example 40: The delivery apparatus of any example herein, particularly any one of examples 38-39, wherein the nosecone comprises one or more flush ports extending along an outer surface of the proximal portion, wherein the flush ports are configured to allow fluid to flow therethrough.

Example 41: The delivery apparatus of any example herein, particularly any one of examples 38-41, wherein the distal portion of the nosecone comprises a plurality of circumferential ribs coaxially along a length of the distal portion and forming a plurality of gaps therebetween.

Example 42: The delivery apparatus of any example herein, particularly example 41, wherein the plurality of gaps between circumferential ribs provide space for each rib to move toward or away from an adjacent rib as a force is applied to the distal portion such that the distal portion bends relative to the proximal portion of the nosecone and the delivery capsule.

Example 43: The delivery apparatus of any example herein, particularly any one of examples 41-42, wherein the distal portion of the nosecone tapers from the proximal portion such that each circumferential rib has a diameter different from each other circumferential rib.

Example 44: The delivery apparatus of any example herein, particularly any one of examples 41-43, wherein each circumferential rib has a diameter less than a diameter of each of the preceding circumferential ribs along the distal portion.

Example 45: The delivery apparatus of any example herein, particularly any one of examples 41-44, wherein the distal portion of the nosecone comprises a conical portion distal to the plurality of circumferential ribs, wherein the conical portion is configured to bend relative to the plurality of circumferential ribs.

Example 46: The delivery apparatus of any example herein, particularly example 45, wherein the conical portion has a diameter less than each of the circumferential ribs.

Example 47: The delivery apparatus of any example herein, particularly any one of examples 38-46, wherein the proximal portion and the distal portion of the nosecone are coupled by a joint comprising a projection received in a cavity, and wherein one of the proximal portion and the distal portion comprise the projection and the other of the proximal portion and the distal portion comprise the cavity.

Example 48: The delivery apparatus of any example herein, particularly any one of examples 38-47, wherein the distal rim encircles a midsection of the nosecone formed at an interface of the proximal portion and the distal portion of the nosecone when the delivery capsule is in the closed position.

Example 49: The delivery apparatus of any example herein, particularly any one of examples 38-48, wherein the distal rim encircles an outer surface of the distal portion when the delivery capsule is in the closed position.

Example 50: The delivery apparatus of any example herein, particularly any one of examples 41-49, wherein the distal rim encircles one or more of the circumferential ribs when the delivery capsule is in the closed position.

Example 51: The delivery apparatus of any example herein, particularly any one of examples 41-50, wherein the distal rim encircles one or more of the gaps between adjacent circumferential ribs when the delivery capsule is in the closed position.

Example 52: The delivery apparatus of any example herein, particularly any one of examples 38-51, wherein the distal rim encircles an outer surface of the proximal portion when the delivery capsule is in the closed position.

Example 53: The delivery apparatus of any example herein, particularly any one of examples 38-52, wherein the distal rim is flexible relative to the main body of the delivery capsule such that the distal rim is configured to move with the distal portion of the nosecone as the distal portion bends relative to the delivery capsule.

Example 54: The delivery apparatus of any example herein, particularly any one of examples 38-53, wherein an inner surface of the delivery capsule transitions from a first diameter to a second diameter when the delivery capsule is in a closed position.

Example 55: The delivery apparatus of any example herein, particularly any one of examples 38-54, wherein the proximal portion and the distal portion of the nosecone comprise a guidewire lumen sized to receive and pass a guidewire therethrough.

Example 56: The delivery apparatus of any example herein, particularly example 55, wherein the guidewire lumen of the distal portion is configured to bend upon bending of the distal portion relative to the proximal portion and the delivery capsule.

Example 57: The delivery apparatus of any example herein, particularly any one of examples 38-56, wherein the proximal portion and the distal portion of the nosecone are fixed to the distal portion of the first shaft.

Example 58: The delivery apparatus of any example herein, particularly any one of examples 38-57, wherein the delivery capsule comprises an annular band configured to limit axial movement of the delivery capsule relative to the nosecone when the delivery capsule is in a closed position.

Example 59: A delivery apparatus for an expandable prosthetic heart valve, the delivery apparatus comprising: a handle; a first shaft having a distal portion and proximal portion coupled to the handle; a nosecone coupled to the distal portion of the first shaft, the nosecone comprising a proximal portion and a distal portion coupled to the proximal portion by a joint comprising a projection received in a cavity, wherein the distal portion of the nosecone comprises a plurality of circumferential grooves along a portion of the distal portion, wherein the proximal portion is formed of a first material that is relatively harder than a second material forming the distal portion; and a second shaft extending over the first shaft; and a delivery capsule coupled to a distal end portion of the second shaft, wherein the delivery capsule comprises a tapered-distal tip portion and a main body configured to receive the proximal portion of the nosecone and retain an expandable prosthetic heart valve in a radially-compressed configuration when the delivery capsule is in a closed position; wherein the tapered-distal tip portion is flexible relative to the main body and configured to extend over one or more of the circumferential grooves of the distal portion and an midsection of the nosecone formed at an interface of the proximal portion and the distal portion of the nosecone when the delivery capsule is in the closed position.

Example 60: A method for delivering an expandable prosthetic heart valve, the method comprising: advancing a delivery apparatus into a native vasculature of a patient, wherein the delivery apparatus comprises a nosecone coupled to a distal end portion of a shaft and an expandable prosthetic heart valve mounted in a radially-compressed configuration around the distal end portion of the shaft, wherein the nosecone comprises proximal portion and a distal portion coupled to the proximal portion, and wherein the distal portion comprises a first material and the proximal portion comprises a second material, wherein the first material is softer than the second material; and urging a distal portion of the nosecone against a vasculature wall of the patient to cause the distal portion to bend relative to the proximal portion.

Example 61: A method for delivering an expandable prosthetic heart valve, the method comprising: advancing into a native vasculature of a patient a delivery apparatus comprising an expandable prosthetic heart valve mounted in a radially-compressed configuration around a distal portion of a first shaft and retained in a delivery capsule of a second shaft extending over the first shaft, such that a distal portion of a nosecone of the delivery apparatus contacts a vasculature wall of the patient, wherein contact between the nosecone and the vasculature wall of the patient causes the distal portion of the nosecone to flexibly curve relative to a proximal portion of the nosecone; wherein the delivery capsule comprises a tapered-distal tip extending over a portion of the distal portion of the nosecone such that the tapered-distal tip minimizes formation of a gap between the nosecone and delivery capsule as the distal portion flexibly curves.

Example 62: A method for implanting an expandable prosthetic heart valve into an aortic annulus of a patient, the method comprising: advancing an expandable prosthetic heart valve and a distal portion of a first shaft and a second shaft of a delivery apparatus into an aorta of the patient such that a distal portion of a nosecone of the delivery apparatus contacts a wall of the aorta, wherein the expandable prosthetic heart valve is mounted in a radially-compressed configuration around a distal portion of the first shaft and retained within a delivery capsule along a distal section of the second shaft, and wherein a proximal portion of the nosecone is coupled to the distal portion of the first shaft and a distal rim of the delivery capsule extends over a portion of the distal portion of the nosecone; wherein contact between the nosecone and the wall of the aorta causes the distal portion of the nosecone to flexibly curve relative to the proximal portion of the nosecone and the delivery capsule, and the distal rim minimizes formation of a gap between the nosecone and the delivery capsule as the distal portion of the nosecone flexibly curves; inserting the delivery capsule and the expandable prosthetic heart valve into a native annulus of the patient such that the nosecone extends through the aortic annulus and into a left ventricle of the patient; retracting the second shaft; and expanding the prosthetic heart valve from a radially-compressed configuration to a radially-expanded configuration within the native annulus.

Example 63: An expandable prosthetic heart valve delivery assembly comprising: a delivery apparatus comprising a handle, a shaft comprising a distal portion and a proximal portion coupled to the handle, and a nosecone coupled to the distal portion of the shaft, the nosecone comprising a proximal portion and a distal portion coupled to the proximal portion; and an expandable prosthetic heart valve mounted in a radially-compressed configuration around the distal portion of the shaft; wherein the proximal portion is formed of a first material that is relatively harder than a second material forming the distal portion, and wherein the distal portion of the nosecone is configured to flexibly curve relative the proximal portion of the nosecone and the expandable prosthetic heart valve.

Example 64: An expandable prosthetic heart valve delivery assembly comprising: a delivery apparatus comprising a handle, a shaft comprising a distal portion and a proximal portion coupled to the handle, and a nosecone coupled to the distal portion of the shaft, the nosecone comprising a proximal portion and a distal portion coupled to the proximal portion; and an expandable prosthetic heart valve mounted in a radially-compressed configuration around the distal portion of the shaft; wherein the distal portion of the nosecone comprises a material relatively softer than a material of the proximal portion and a plurality of circumferential grooves aligned coaxially along the distal portion of the nosecone such that the body of the distal portion is configured to flexibly curve relative to the proximal portion of the nosecone.

Example 65: An expandable prosthetic heart valve delivery assembly comprising: a delivery apparatus comprising a handle, a shaft comprising a distal portion and a proximal portion coupled to the handle, and a nosecone coupled to the distal portion of the shaft, the nosecone comprising a proximal portion and a distal portion coupled to the proximal portion by a projection received in a cavity, wherein the proximal portion is formed of a first material that is relatively harder than a second material forming the distal portion; and an expandable prosthetic heart valve mounted in a radially-compressed configuration around the distal portion of the shaft; wherein the distal portion is configured to flexibly curve relative the proximal portion of the nosecone, and wherein one of the proximal portion and the distal portion comprises the projection and the other of the proximal portion and the distal portion comprises the cavity.

Example 66: An expandable prosthetic heart valve delivery assembly comprising: a delivery apparatus comprising a handle, a first shaft, and a second shaft extending over the first shaft, each shaft comprising a distal portion and a proximal portion coupled to the handle, the distal portion of the second shaft having a delivery capsule comprising a main body and a tapered-distal tip axially extending from the main body; and an expandable prosthetic heart valve mounted in a radially-compressed configuration around the distal portion of the first shaft and within the delivery capsule of the second shaft; wherein the tapered-distal tip of the delivery capsule extends circumferentially around a tapered portion of a nosecone coupled to the distal portion of the first shaft.

Example 67: An expandable prosthetic heart valve delivery assembly comprising: a delivery apparatus comprising a handle, a nosecone, a first shaft, and a second shaft extending over the first shaft, each shaft comprising a distal portion and a proximal portion coupled to the handle, the nosecone being coupled to the distal portion of the first shaft and the second shaft having a delivery capsule comprising a main body and a tapered-distal tip axially extending from the main body; and an expandable prosthetic heart valve mounted in a radially-compressed configuration around the distal portion of the first shaft and within the delivery capsule of the second shaft; wherein the nosecone comprises a proximal portion and a distal portion coupled to the proximal portion, the proximal portion being received by the delivery capsule and the distal portion of the nosecone being configured to flexibly curve relative the proximal portion of the nosecone and the expandable prosthetic heart valve.

Example 68: The delivery assembly of any example herein, particularly example 67, wherein the tapered-distal tip extends circumferentially around a distal end portion of the second shaft.

Example 69: The delivery assembly of any example herein, particularly example 67, wherein the second shaft comprises a distal edge configured to but against an outer ridge of the proximal portion of the nosecone.

Example 70: An expandable prosthetic heart valve delivery assembly comprising: a delivery apparatus comprising a handle, a nosecone a first shaft, and a second shaft extending over the first shaft, each shaft comprising a distal portion and a proximal portion coupled to the handle, the nosecone being coupled to the distal portion of the first shaft, and the second shaft having a delivery capsule comprising a main body and a tapered-distal tip axially extending from the main body; and an expandable prosthetic heart valve mounted in a radially-compressed configuration around the distal portion of the first shaft and within the delivery capsule of the second shaft; wherein the nosecone comprises a distal portion, a proximal portion coupled to the distal portion, and a flush port extending along and through the surface of the distal portion and the proximal portion, the distal portion of the nosecone being configured to flexibly curve relative to the proximal portion and the delivery capsule and the flush port being configured to allow fluid to flow therethrough; and wherein the tapered-distal tip is configured to extend partially over the distal portion, the proximal portion, and the flush port of the nosecone.

Example 71. A delivery apparatus, delivery assembly or prosthetic valve of any example herein, particularly, any one of examples 1-70, wherein the delivery apparatus, delivery assembly or prosthetic valve is sterilized.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus for an expandable prosthetic heart valve, the delivery apparatus comprising:
a handle;
a shaft having a proximal portion and a distal portion, the proximal portion being coupled to the handle; and
a nosecone coupled to the distal portion of the shaft, the nosecone comprising a proximal portion and a distal portion coupled to the proximal portion, wherein the distal portion is configured to flexibly curve relative to the proximal portion and the proximal portion is formed of a first material that is relatively harder than a second material forming the distal portion.

2. The delivery apparatus of claim 1, wherein the distal portion of the nosecone comprises a plurality of axially spaced circumferential grooves.

3. The delivery apparatus of claim 2, wherein the plurality of circumferential grooves defines a plurality of ribs that move relative to each other when the distal portion flexibly curves relative to the proximal portion.

4. The delivery apparatus of claim 1, wherein the proximal and distal portions of the nosecone are coupled by a projection received in a cavity, wherein one of the proximal portion and the distal portion comprises the projection and the other of the proximal portion and distal portion comprises the cavity.

5. The delivery apparatus of claim 1, wherein the proximal portion comprises an outer surface, an outer ridge extending outwardly from and circumferentially around the outer surface, and one or more flush ports extending through and along the outer surface and outer ridge of the proximal portion.

6. The delivery apparatus of claim 1, wherein the distal portion comprises a first end proximate the proximal portion and a second end distal the first end, and wherein the distal portion tapers from the first end to the second end.

7. The delivery apparatus of claim 1, wherein the shaft comprises a first shaft and the delivery apparatus further comprises a second shaft extending over the first shaft and a delivery capsule coupled to the second shaft, wherein the delivery capsule is configured to receive the proximal portion of the nosecone and retain an expandable prosthetic heart valve in a radially-compressed configuration for delivery into a patient.

8. The delivery apparatus of claim 7, wherein the delivery capsule comprises a tapered distal end portion configured to extend partially over the distal portion of the nosecone.

9. The delivery apparatus of claim 1, wherein the proximal portion of the nosecone comprises a first lumen and the distal portion of the nosecone comprises a second lumen, and wherein the first and second lumens are sized to receive a guidewire therethrough.

10. The delivery apparatus of claim 9, wherein the second lumen is configured to have the same curvature as the distal portion when the distal portion curves relative to the proximal portion of the nosecone.

11. A delivery apparatus for an expandable prosthetic heart valve, the delivery apparatus comprising:
a handle; and
a first shaft and a second shaft extending over the first shaft, each shaft comprising a distal portion and a proximal portion coupled to the handle, the distal portion of the second shaft being coupled to a delivery capsule of the delivery apparatus,
wherein the delivery capsule comprises a main body configured to retain an expandable prosthetic heart valve in a radially-compressed configuration for delivery into a patient and a tapered-distal tip portion axially extending from the main body and tapering in a distal direction.

12. The delivery apparatus of claim 11, wherein a material forming the tapered-distal tip portion has a hardness less than a material forming the main body.

13. The delivery apparatus of claim 11, wherein the tapered-distal tip portion extends circumferentially around a distal portion of the main body.

14. The delivery apparatus of claim 11, wherein the tapered-distal tip portion comprises a cylindrical portion and a tapered portion, and wherein the cylindrical portion extends circumferentially around a distal end portion of the main body and the tapered-distal tip portion tapers in a direction extending distally from the cylindrical portion and forms an opening to the delivery capsule.

15. The delivery apparatus of claim 11, wherein an outer surface of the main body and an outer surface of the tapered-distal tip portion form a continuous outer surface of the delivery capsule.

16. The delivery apparatus of claim 11, wherein an inner surface of the main body and an inner surface of the tapered-distal tip portion form a continuous inner surface of the delivery capsule.

17. The delivery apparatus of claim 11, wherein the main body comprises an outer surface and the tapered-distal tip portion comprises an inner surface, and wherein the outer surface of the main body and the inner surface of the tapered-distal tip portion form an interface where the tapered-distal tip portion extends circumferentially over the main body.

18. The delivery apparatus of claim 17, wherein the delivery capsule comprises at least one radiopaque marker band located at the interface formed by the main body and the tapered-distal tip portion.

19. The delivery apparatus of claim 18, further comprising a nosecone coupled to the distal portion of the first shaft, wherein the delivery capsule is configured to move to a closed position in which the tapered-distal tip portion of the delivery capsule extends along an outer surface of the nosecone.

20. The delivery apparatus of claim 19, wherein the radiopaque marker band is configured to limit relative axial movement between the delivery capsule and the nosecone when the delivery capsule is in a closed position.
